(19) United States
(12) Patent Application Publication
(10) Pub. No.: US 2022/0133699 A1
(43) Pub. Date: May 5, 2022

(54) Antibacterial Compositions

(71) Applicants: Singapore Eye Research Institute, Singapore (SG); SinSa Labs AB, Stockholm (SE)

(72) Inventors: Roger Beuerman (deceased), Singapore (SG); Urban Olson, Stockholm (SE)

(21) Appl. No.: 17/431,077

(22) PCT Filed: Feb. 14, 2020
(86) PCT No.: PCT/US2020/018397

§ 371 Date: Aug. 13, 2021

Related U.S. Application Data
(60) Provisional application No. 62/805,621, filed on Feb. 14, 2019.

(51) Int. Cl. A61K 31/431; A61K 31/407; A61K 45/06; A61P 31/04

(52) U.S. Cl. CPC A61K 31/431; A61P 31/04; A61K 45/06; A61K 31/407

(57) ABSTRACT

Provided herein are methods of treating subjects having a bacterial infection by administering a peptide multimer and a carbapenem antibiotic. Also provided herein are methods of eliminating or inhibiting bacteria with a peptide multimer and a carbapenem antibiotic. Also provided herein are antibacterial compositions that include a peptide multimer and a carbapenem antibiotic.

### BACKGROUND

Amino acids both natural and non-natural, assembled into configurations of non-natural branched peptides of multimeric form having antimicrobial activity based on peptide sequences have been described in the art. Hydrophobicity and the net positive charge of branched peptide multimers contribute to their interaction with and disruption of the Gram-negative bacteria cell wall and inner cell membrane.

Carbapenems are a class of antibiotic agents commonly used for the treatment of severe or high-risk bacterial infections. Similar to penicillins and cephalosporins, carbapenems are members of the beta lactam class of antibiotics, which kill bacteria by binding to penicillin-binding proteins, thus inhibiting bacterial cell wall synthesis. Of the many hundreds of different β-lactams, however, carbapenems possess the broadest spectrum of activity and greatest potency against Gram-positive and Gram-negative bacteria. Given the efficacy of this class of antibiotics, these agents are often reserved for known or suspected multidrug-resistant (MDR) bacterial infections.

Carbapenem antibiotics are an environmental stress on bacteria that select for those bacteria that have undergone a genetic change that results in antibiotic resistance. Not only is it desirable to develop new technologies to overcome bacterial resistance in general, but lowering the concentration of antibiotics required to kill bacteria or prevent their growth will reduce the development of future resistance.

### SUMMARY

The present disclosure generally relates to methods of treating a bacterial infection in a subject by administering a peptide multimer and a carbapenem antibiotic (e.g., simultaneously or sequentially), methods of inhibiting bacterial growth using such a peptide multimer and a carbapenem antibiotic, and antibacterial compositions that include a peptide multimer and a carbapenem antibiotic. Other embodiments of the invention will be described in more detail herein.

Provided herein are methods of inhibiting growth of a bacterium comprising: contacting the bacterium with an effective amount of: a peptide multimer of the formula (U)nBmZj, wherein U is a peptide comprising RGRKVVRR, wherein n≥2, m≥1, and j≥0, wherein each B comprises at least one amino acid having at least two amine groups, wherein Z comprises any amino acid, and wherein the multimer is branched at the terminal BmZj residues; and a carbapenem antibiotic or a pharmaceutically acceptable salt thereof.

In some embodiments, the growth of the bacterium is inhibited by at 2-fold as compared to a reference bacterium that is not contacted with the peptide multimer and the carbapenem antibiotic.

In some embodiments, the peptide multimer is present in an amount that is lower than the amount of the peptide multimer required to provide the same level of growth inhibition of the bacterium in the absence of the carbapenem antibiotic. In some embodiments, the peptide multimer is present in an amount that is lower than about 4 μg/mL.

In some embodiments, the carbapenem antibiotic, or a pharmaceutically acceptable salt thereof, is selected from the group consisting of: meropenem, imipenem, doripenem, eratapenem, panipenem (betamipron), biapenem, tebipenem, razupenem (PZ-601), lenapenem, tomopenem, thienamycin (thienpenem), and combinations thereof.

In some embodiments, the carbapenem antibiotic, or a pharmaceutically acceptable salt thereof, is present in an amount that is lower than the amount of the carbapenem antibiotic, or a pharmaceutically acceptable salt thereof, required to provide the same level of growth inhibition of the bacterium in the absence of the peptide multimer.

In some embodiments, the carbapenem antibiotic comprises meropenem. In some embodiments, the meropenem is present in an amount that is lower than about 4 μg/mL.

In some embodiments, the carbapenem antibiotic comprises imipenem. In some embodiments, the imipenem is present in an amount that is lower than about 4 μg/mL.

In some embodiments, the carbapenem antibiotic comprises doripenem. In some embodiments, the doripenem is present in an amount that is lower than about 4 μg/mL.

In some embodiments, the bacterium is Klebsiella pneumonia. In some embodiments, the Klebsiella pneumonia bacterium is Klebsiella pneumonia strain 8852, Klebsiella pneumonia strain 27025, or Klebsiella pneumonia strain 24076.

In some embodiments, the bacterium is Acinetobacter baumannii. In some embodiments, the Acinetobacter baumannii bacterium is Acinetobacter baumannii strain 1010.

Also provided herein are methods of treating a bacterial infection in a subject comprising administering to the subject a therapeutically effective amount of: a peptide multimer of the formula (U)nBmZj, wherein U is a peptide comprising RGRKVVRR, wherein n≥2, m≥1, and j≥0, wherein each B comprises at least one amino acid having at least two amine groups, wherein Z comprises any amino acid, and wherein the multimer is branched at the terminal BmZj residues; and a carbapenem antibiotic or a pharmaceutically acceptable salt thereof.

In some embodiments, the number of bacteria is decreased in the subject by at least 2-fold as compared the number of bacteria in a reference subject that is not administered the peptide multimer and the carbapenem antibiotic.

In some embodiments, the peptide multimer is present in an amount that is lower than the amount of the peptide multimer required to provide the same level of bacterial inhibition when the peptide multimer is administered to the subject in the absence of the carbapenem antibiotic.

In some embodiments, wherein the peptide multimer is administered in an amount from about 0.1 ug/ml and about 20 ug/ml.

In some embodiments, the carbapenem antibiotic, or a pharmaceutically acceptable salt thereof, is selected from the group consisting of: meropenem, imipenem, doripenem, eratapenem, panipenem (betamipron), biapenem, tebipenem, razupenem (PZ-601), lenapenem, tomopenem, thienamycin (thienpenem), and combinations thereof.

In some embodiments, wherein the carbapenem antibiotic, or a pharmaceutically acceptable salt thereof, is administered in an amount that is lower than the amount of the carbapenem antibiotic, or a pharmaceutically acceptable salt thereof, required to provide the same level of bacterial inhibition when the carbapenem antibiotic is administered to a reference subject in the absence of the peptide multimer.

In some embodiments, wherein the carbapenem antibiotic comprises meropenem. In some embodiments, the meropenem is administered in an amount that is lower than about 4 μg/mL.

In some embodiments, the carbapenem antibiotic comprises imipenem. In some embodiments, the imipenem is administered in an amount that is lower than about 4 μg/mL.

In some embodiments, the carbapenem antibiotic comprises doripenem. In some embodiments, the doripenem is administered in an amount that is lower than about 4 μg/mL.

In some embodiments, the bacterial infection is caused by Klebsiella pneumonia. In some embodiments, the Klebsiella pneumonia bacterium is Klebsiella pneumonia strain 8852, Klebsiella pneumonia strain 27025, or Klebsiella pneumonia strain 24076.

In some embodiments, the bacterial infection is caused by Acinetobacter baumannii. In some embodiments, the Acinetobacter baumannii bacterium is Acinetobacter baumannii strain 1010.

In some embodiments, the peptide multimer and the carbapenem antibiotic are administered to the subject simultaneously.

In some embodiments, the peptide multimer and the carbapenem antibiotic are administered to the subject sequentially.

Also provided herein are antibacterial compositions comprising: a peptide multimer of the formula (U)nBmZj, wherein U is a peptide comprising RGRKVVRR, wherein n≥2, m≥1, and j≥0, wherein each B comprises at least one amino acid having at least two amine groups, wherein Z comprises any amino acid, and wherein the multimer is branched at the terminal BmZj residues; and a carbapenem antibiotic or a pharmaceutically acceptable salt thereof.

In some embodiments, the carbapenem antibiotic, or a pharmaceutically acceptable salt thereof, is selected from the group consisting of: meropenem, imipenem, doripenem, eratapenem, panipenem (betamipron), biapenem, tebipenem, razupenem (PZ-601), lenapenem, tomopenem, thienamycin (thienpenem), and combinations thereof.

In some embodiments, the peptide multimer comprises (RGRKVVRR)₂KK.

In some embodiments, the carbapenem antibiotic comprises meropenem, and wherein the peptide multimer comprises (RGRKVVRR)₂KK.

In some embodiments, the carbapenem antibiotic comprises imipenem, and wherein the peptide multimer comprises (RGRKVVRR)₂KK.

In some embodiments, the carbapenem antibiotic comprises doripenem, and wherein the peptide multimer comprises (RGRKVVRR)₂KK.

In some embodiments, the peptide multimers and the carbapenem antibiotic are formulated for use in treating a bacterial infection.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Structure of B2088

FIG. 2. Structure of B4010

### DETAILED DESCRIPTION

As used herein, the word “a” before a noun represents one or more of the particular noun. For example, the phrase “a peptide multimer” encompasses “one or more peptide multimers.” Similarly, the phrase “a carbapenem antibiotic” encompasses “one or more carbapenem antibiotics.”

As used herein, the term “about” means approximately, in the region of, roughly, or around. When used in conjunction with a numerical range, the term “about” modifies that range by extending the boundaries above and below the numerical values set forth. In general, the term “about” is used herein to modify a numerical value above and below the stated value by a variance of 10%.

As used herein, the term “amino acid” refers to naturally and non-naturally occurring L- and D-amino acids, peptidomimetic amino acids, and non-standard amino acids that are not made by a standard machinery or are only found in proteins after post-translational modification or as metabolic intermediates.

The term “antimicrobial”, when used herein in reference to an agent or a composition, refers to the property of eliminating (e.g., killing) bacteria, reducing or preventing bacterial growth, or treating diseases caused by bacteria.

As used herein, the phrase “non-proteogenic amino acid” refers to an amino acid that is not one of the 20 amino acids that is normally found in naturally-occurring proteins.

As used herein, the term “subject” refers to a vertebrate, including any member of the class mammalia, including humans, domestic and farm animals, and zoo, sports or pet animals, such as mouse, rabbit, pig, sheep, goat, cattle, horse (e.g., race horse), and higher primates. In some embodiments, the subject is a human. In some embodiments, the subject has a disease. In some embodiments, the subject has a bacterial infection.

The term “synergy” or “synergistic” is used herein to mean that the effect of the combination of the two or more therapeutic agents of the combination therapy is greater than the sum of the effect of each agent when administered alone. A “synergistic amount” or “synergistically effective amount” is an amount of the combination of the two or more combination partners that results in a synergistic effect, as “synergistic” is defined herein. Determining a synergistic interaction between two or more combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions and reduction of unwanted side effects.

### Methods of Treating

Provided herein are methods of treating a subject having a bacterial infection (e.g., a bacterial infection caused a bacterium of any of the variety of bacterial genii or species provided herein), the method comprising administering to the subject a peptide multimer and a carbapenem antibiotic. In some embodiments, any of the variety of peptide multimers and carbapenem antibiotics described herein are administered to a subject having a bacterial infection such that the bacterial infection is treated. For example, any of the variety of peptide multimers and carbapenem antibiotics described herein can be administered to a subject having a bacterial infection, and the number of bacteria present in the subject can be reduced. In some embodiments, after treatment with any of the variety of peptide multimers and carbapenem antibiotics described herein, the number of bacteria present in the subject is reduced by at least 50%, at least 55%, at least 60%, at least 65%, at least 70%, at least 75%, at least 80%, at least 85%, at least 90%, at least 95%, at least 96%, at least 97%, at least 98%, at least 99%, or more. In some embodiments, after treatment with any of the variety of peptide multimers and carbapenem antibiotics described herein, the reduction in the number of bacteria present in the subject is greater than the reduction in the number of bacteria in a reference subject having the same bacterial infection who is treated with either the peptide multimer or the carbapenem antibiotic alone. In some embodiments, after treatment with any of the variety of peptide multimers and carbapenem antibiotics described herein, the number of bacteria present in the subject is reduced by a factor of at least 2-fold, at least 3-fold, at least 4-fold, at least 5-fold, at least 6-fold, at least 7-fold, at least 8-fold, at least 9-fold, at least 10-fold, or more as compared to the number of bacteria present in a reference subject having the same bacterial infection who is treated with either the peptide multimer or the carbapenem antibiotic alone. Additionally or alternatively, any of the variety of peptide multimers and carbapenem antibiotics described herein can be administered to a subject having a bacterial infection, and the growth of the bacteria present in the subject can be reduced or prevented. In some embodiments, after treatment with any of the variety of peptide multimers and carbapenem antibiotics described herein, the growth of bacteria present in the subject is reduced by at least 50%, at least 55%, at least 60%, at least 65%, at least 70%, at least 75%, at least 80%, at least 85%, at least 90%, at least 95%, at least 96%, at least 97%, at least 98%, at least 99%, or more. In some embodiments, after treatment with any of the variety of peptide multimers and carbapenem antibiotics described herein, the reduction in the growth of bacteria present in the subject is greater than the reduction in the growth of bacteria in a reference subject having the same bacterial infection who is treated with either the peptide multimer or the carbapenem antibiotic alone. In some embodiments, after treatment with any of the variety of peptide multimers and carbapenem antibiotics described herein, the growth of bacteria present in the subject is reduced by a factor of at least 2-fold, at least 3-fold, at least 4-fold, at least 5-fold, at least 6-fold, at least 7-fold, at least 8-fold, at least 9-fold, at least 10-fold, or more as compared to the growth of bacteria present in a reference subject having the same bacterial infection who is treated with either the peptide multimer or the carbapenem antibiotic alone. In some embodiments, subjects treated with any of the variety of peptide multimers and carbapenem antibiotics described herein have better clinical outcomes (e.g., improved better clinical outcomes, e.g., significantly improved better clinical outcomes) as compared to a reference subject having the same bacterial infection who is treated with either the peptide multimer or the carbapenem antibiotic alone, regardless of whether the bacterium produces beta-lactamase enzymes.

In some embodiments, a subject having a bacterial infection (e.g., a bacterial infection caused a bacterium of any of the variety of bacterial genii or species provided herein) that is treated with any of the variety of peptide multimers and carbapenem antibiotics described herein has been previously treated with a carbapenem antibiotic and has developed resistance to that carbapenem antibiotic. In some embodiments, a subject who has a bacterial infection and who has developed resistance to a carbapenem antibiotic responds to treatment with the peptide multimer and carbapenem antibiotic, but does not respond or responds less well to treatment with the carbapenem antibiotic alone. In some embodiments, a subject who has a bacterial infection and who has developed resistance to a carbapenem antibiotic is administered any of the variety of peptide multimers and carbapenem antibiotics described herein such that the number of bacteria present in the subject is reduced by at least 50%, at least 55%, at least 60%, at least 65%, at least 70%, at least 75%, at least 80%, at least 85%, at least 90%, at least 95%, at least 96%, at least 97%, at least 98%, at least 99%. In some embodiments, after treatment with any of the variety of peptide multimers and carbapenem antibiotics described herein, the reduction in the number of bacteria present in the subject having resistance to a carbapenem antibiotic is greater than the reduction in the number of bacteria present in a reference subject having the same bacterial infection and resistance to the carbapenem antibiotic, and who is treated with the carbapenem antibiotic alone. In some embodiments, a subject who has a bacterial infection and who has developed resistance to a carbapenem antibiotic is administered any of the variety of peptide multimers and carbapenem antibiotics described herein such that the number of bacteria present in the subject is reduced by a factor of at least 2-fold, at least 3-fold, at least 4-fold, at least 5-fold, at least 6-fold, at least 7-fold, at least 8-fold, at least 9-fold, at least 10-fold, or more as compared to the number of bacteria present in a reference subject having the same bacterial infection and resistance to the carbapenem antibiotic, and who is treated with a the carbapenem antibiotic alone. Additionally or alternatively, any of the variety of peptide multimers and carbapenem antibiotics described herein can be administered to a subject having a bacterial infection and who has developed resistance to the carbapenem antibiotic, and the growth of the bacteria present in the subject can be reduced or prevented. In some embodiments, a subject who has a bacterial infection and who has developed resistance to a carbapenem antibiotic is administered any of the variety of peptide multimers and carbapenem antibiotics described herein such that the growth of bacteria present in the subject is reduced by at least 50%, at least 55%, at least 60%, at least 65%, at least 70%, at least 75%, at least 80%, at least 85%, at least 90%, at least 95%, at least 96%, at least 97%, at least 98%, at least 99%. In some embodiments, after treatment with any of the variety of peptide multimers and carbapenem antibiotics described herein, the reduction in the growth of bacteria present in the subject having carbapenem resistance is greater than the reduction in the growth of bacteria in a reference subject having the same bacterial infection and resistance to the carbapenem antibiotic, and who is treated with the carbapenem antibiotic alone. In some embodiments, a subject who has a bacterial infection and who has developed resistance to a carbapenem antibiotic is administered any of the variety of peptide multimers and carbapenem antibiotics described herein such that the growth of bacteria present in the subject is reduced by at least 2-fold, at least 3-fold, at least 4-fold, at least 5-fold, at least 6-fold, at least 7-fold, at least 8-fold, at least 9-fold, at least 10-fold, or more as compared to the number of bacteria present in a reference subject having the same bacterial infection and resistance to the carbapenem antibiotic, and who is treated with the carbapenem antibiotic alone. In some embodiments, subjects who have developed resistance to a carbapenem antibiotic and who are treated with any of the variety of peptide multimers and carbapenem antibiotics described herein that include a peptide multimer and a carbapenem antibiotic have better clinical outcomes (e.g., improved better clinical outcomes, e.g., significantly improved better clinical outcomes) as compared to a reference subject having the same bacterial infection and resistance to the same carbapenem antibiotic who is treated with the carbapenem antibiotic alone, regardless of whether the bacterium produces beta-lactamase enzymes.

In some embodiments, a subject having a bacterial infection (e.g., a bacterial infection caused a bacterium of any of the variety of bacterial genii or species provided herein) that is treated with any of the variety of peptide multimers and carbapenem antibiotics described herein has been previously treated with a peptide multimer and has developed resistance to that peptide multimer. In some embodiments, a subject who has a bacterial infection and who has developed resistance to a peptide multimer responds to treatment with the peptide multimer and carbapenem antibiotic, but does not respond or responds less well to treatment with the peptide multimer alone. In some embodiments, a subject who has a bacterial infection and who has developed resistance to a peptide multimer is administered any of the variety of peptide multimers and carbapenem antibiotics described herein such that the number of bacteria present in the subject is reduced by at least 50%, at least 55%, at least 60%, at least 65%, at least 70%, at least 75%, at least 80%, at least 85%, at least 90%, at least 95%, at least 96%, at least 97%, at least 98%, at least 99%. In some embodiments, after treatment with any of the variety of peptide multimers and carbapenem antibiotics described herein, the reduction in the number of bacteria present in the subject having resistance to a peptide multimer is greater than the reduction in the number of bacteria present in a reference subject having the same bacterial infection and resistance to the peptide multimer, and who is treated with a the peptide multimer alone. In some embodiments, a subject who has a bacterial infection and who has developed resistance to a peptide multimer is administered any of the variety of peptide multimers and carbapenem antibiotics described herein such that the number of bacteria present in the subject is reduced by a factor of at least 2-fold, at least 3-fold, at least 4-fold, at least 5-fold, at least 6-fold, at least 7-fold, at least 8-fold, at least 9-fold, at least 10-fold, or more as compared to the number of bacteria present in a reference subject having the same bacterial infection and resistance to the peptide multimer, and who is treated the peptide multimer alone. Additionally or alternatively, any of the variety of peptide multimers and carbapenem antibiotics described herein can be administered to a subject having a bacterial infection and who has developed resistance to the peptide multimer, and the growth of the bacteria present in the subject can be reduced or prevented. In some embodiments, a subject who has a bacterial infection and who has developed resistance to a peptide multimer is administered any of the variety of peptide multimers and carbapenem antibiotics described herein such that the growth of bacteria present in the subject is reduced by at least 50%, at least 55%, at least 60%, at least 65%, at least 70%, at least 75%, at least 80%, at least 85%, at least 90%, at least 95%, at least 96%, at least 97%, at least 98%, at least 99%, or more. In some embodiments, after treatment with any of the variety of peptide multimers and carbapenem antibiotics described herein, the reduction in the growth of bacteria present in the subject having peptide multimer resistance is greater than the reduction in the growth of bacteria in a reference subject having the same bacterial infection and resistance to the peptide multimer, and who is treated with the peptide multimer alone. In some embodiments, a subject who has a bacterial infection and who has developed resistance to a peptide multimer is administered any of the variety of peptide multimers and carbapenem antibiotics described herein such that the growth of bacteria present in the subject is reduced by at least 2-fold, at least 3-fold, at least 4-fold, at least 5-fold, at least 6-fold, at least 7-fold, at least 8-fold, at least 9-fold, at least 10-fold, or more as compared to the number of bacteria present in a reference subject having the same bacterial infection and resistance to the peptide multimer, and who is treated with the peptide multimer alone. In some embodiments, subjects who have developed resistance to a peptide multimer and who are treated with any of the variety of peptide multimers and carbapenem antibiotics described herein have better clinical outcomes (e.g., improved better clinical outcomes, e.g., significantly improved better clinical outcomes) as compared to a reference subject having the same bacterial infection and resistance to the same peptide multimer who is treated with the peptide multimer alone, regardless of whether the bacterium produces beta-lactamase enzymes.

In some embodiments, a subject who has a bacterial infection (e.g., a subject who has a bacterial infection and has not developed carbapenem antibiotic or peptide multimer resistance, or a subject who has a bacterial infection and has developed carbapenem antibiotic or peptide multimer resistance) is treated with any of the variety of peptide multimers and carbapenem antibiotics (e.g., any of the variety of carbapenem antibiotics described herein or known in the art) provided herein. In some embodiments, a peptide multimer is a peptide dimer. Non limiting examples of peptide dimers and a carbapenem antibiotics that can be administered include: B2088 (e.g., lipidated or non-lipidated) and meropenem, B2088 (e.g., lipidated or non-lipidated) and imipenem, B2088 (e.g., lipidated or non-lipidated) and doripenem, B2088 (e.g., lipidated or non-lipidated) and eratapenem, B2088 (e.g., lipidated or non-lipidated) and panipenem, B2088 (e.g., lipidated or non-lipidated) and biapenem, B2088 (e.g., lipidated or non-lipidated) and tebipenem, B2088 (e.g., lipidated or non-lipidated) and razupenem, B2088 (e.g., lipidated or non-lipidated) and lenapenem, B2088 (e.g., lipidated or non-lipidated) and tomopenem, and B2088 (e.g., lipidated or non-lipidated) and tomopenem.

Other non-limiting examples of peptide dimers and carbapenem antibiotics that can be administered to a subject include: a peptide dimer (e.g., lipidated or non-lipidated) having at least one monomer subunit (e.g., two monomer subunits) of any one of SEQ ID NO: 20-46 and meropenem, a peptide dimer (e.g., lipidated or non-lipidated) having at least one monomer subunit (e.g., two monomer subunits) of any one of SEQ ID NO: 20-46 and imipenem, a peptide dimer (e.g., lipidated or non-lipidated) having at least one monomer subunit (e.g., two monomer subunits) of any one of SEQ ID NO: 20-46 and doripenem, a peptide dimer (e.g., lipidated or non-lipidated) having at least one monomer subunit (e.g., two monomer subunits) of any one of SEQ ID NO: 20-46 and eratapenem, a peptide dimer (e.g., lipidated or non-lipidated) having at least one monomer subunit (e.g., two monomer subunits) of any one of SEQ ID NO: 20-46 and panipenem, a peptide dimer (e.g., lipidated or non-lipidated) having at least one monomer subunit (e.g., two monomer subunits) of any one of SEQ ID NO: 20-46 and biapenem, a peptide dimer (e.g., lipidated or non-lipidated) having at least one monomer subunit (e.g., two monomer subunits) of any one of SEQ ID NO: 20-46 and tebipenem, a peptide dimer (e.g., lipidated or non-lipidated) having at least one monomer subunit (e.g., two monomer subunits) of any one of SEQ ID NO: 20-46 and razupenem, a peptide dimer (e.g., lipidated or non-lipidated) having at least one monomer subunit (e.g., two monomer subunits) of any one of SEQ ID NO: 20-46 and lenapenem, a peptide dimer (e.g., lipidated or non-lipidated) having at least one monomer subunit (e.g., two monomer subunits) of any one of SEQ ID NO: 20-46 and tomopenem, and a peptide dimer (e.g., lipidated or non-lipidated) having at least one monomer subunit (e.g., two monomer subunits) of any one of SEQ ID NO: 20-46 and tomopenem. In some embodiments, the two peptide monomer subunits of the peptide dimer are the same. In some embodiments, the two peptide monomer subunits of the peptide dimer are different.

In some embodiments, a subject who has a bacterial infection (e.g., a subject who has a bacterial infection and has not developed carbapenem antibiotic or peptide multimer resistance, or a subject who has a bacterial infection and has developed carbapenem antibiotic or peptide multimer resistance) is treated with any of the variety of peptide multimers and carbapenem antibiotics (e.g., any of the variety of carbapenem antibiotics described herein or known in the art) provided herein. In some embodiments, a peptide multimer is a peptide tetramer. Non limiting examples of peptide tetramers and a carbapenem antibiotics that can be administered include: include: a peptide tetramer (e.g., lipidated or non-lipidated) having at least one monomer subunit (e.g., one, two, three, or four monomer subunits) of any one of SEQ ID NO: 20-46 and meropenem, a peptide tetramer (e.g., lipidated or non-lipidated) having at least one monomer subunit (e.g., one, two, three, or four monomer subunits) of any one of SEQ ID NO: 20-46 and imipenem, a peptide tetramer (e.g., lipidated or non-lipidated) having at least one monomer subunit (e.g., one, two, three, or four monomer subunits) of any one of SEQ ID NO: 20-46 and doripenem, a peptide tetramer (e.g., lipidated or non-lipidated) having at least one monomer subunit (e.g., one, two, three, or four monomer subunits) of any one of SEQ ID NO: 20-46 and eratapenem, a peptide tetramer (e.g., lipidated or non-lipidated) having at least one monomer subunit (e.g., one, two, three, or four monomer subunits) of any one of SEQ ID NO: 20-46 and panipenem, a peptide tetramer (e.g., lipidated or non-lipidated) having at least one monomer subunit (e.g., one, two, three, or four monomer subunits) of any one of SEQ ID NO: 20-46 and biapenem, a peptide tetramer (e.g., lipidated or non-lipidated) having at least one monomer subunit (e.g., one, two, three, or four monomer subunits) of any one of SEQ ID NO: 20-46 and tebipenem, a peptide tetramer (e.g., lipidated or non-lipidated) having at least one monomer subunit (e.g., one, two, three, or four monomer subunits) of any one of SEQ ID NO: 20-46 and razupenem, a peptide tetramer (e.g., lipidated or non-lipidated) having at least one monomer subunit (e.g., one, two, three, or four monomer subunits) of any one of SEQ ID NO: 20-46 and lenapenem, a peptide tetramer (e.g., lipidated or non-lipidated) having at least one monomer subunit (e.g., one, two, three, or four monomer subunits) of any one of SEQ ID NO: 20-46 and tomopenem, and a peptide tetramer (e.g., lipidated or non-lipidated) having at least one monomer subunit (e.g., one, two, three, or four monomer subunits) of any one of SEQ ID NO: 20-46 and tomopenem. In some embodiments, at least two (e.g., 2, 3 or 4) peptide monomer subunits of the peptide tetramer are the same. In some embodiments, at least two (e.g., 2, 3 or 4) peptide monomer subunits of the peptide tetramer are different.

In some embodiments, a subject who has a bacterial infection (e.g., a subject who has a bacterial infection and has not developed carbapenem antibiotic or peptide multimer resistance, or a subject who has a bacterial infection and has developed carbapenem antibiotic or peptide multimer resistance) is treated with any of the variety of peptide multimers (e.g., one or more, e.g., 1, 2, 3, 4, 5, 6, 7, 8, 9, 10, or more of any of the variety of peptide multimers described herein or known in the art) and a single carbapenem antibiotic (e.g., any of the variety of carbapenem antibiotics described herein or known in the art). In some embodiments, a subject is treated with two or more (e.g., 2, 3, 4, 5, 6, 7, 8, 9, 10, or more) carbapenem antibiotics (e.g., any of the variety of carbapenem antibiotics disclosed herein) and a single or two or more (e.g., 2, 3, 4, 5, 6, 7, 8, 9, 10, or more) peptide multimers (e.g., any of the variety of peptide multimers disclosed herein).

In some embodiments, a subject who has a bacterial infection (e.g., a subject who has a bacterial infection and has not developed carbapenem antibiotic or peptide multimer resistance, or a subject who has a bacterial infection and has developed carbapenem antibiotic or peptide multimer resistance) is treated with any of the variety of carbapenem antibiotics (e.g., one or more, e.g., 1, 2, 3, 4, 5, 6, 7, 8, 9, 10, or more of any of the variety of carbapenem antibiotics described herein or known in the art) and a single peptide multimer (e.g., any of the variety of peptide multimers described herein or known in the art). In some embodiments, a subject is treated with two or more (e.g., 2, 3, 4, 5, 6, 7, 8, 9, 10, or more) peptide multimers (e.g., any of the variety of peptide multimers disclosed herein) and a single or two or more (e.g., 2, 3, 4, 5, 6, 7, 8, 9, 10, or more) carbapenem antibiotics (e.g., any of the variety of carbapenem antibiotics disclosed herein).

In some embodiments, the peptide multimer and the carbapenem antibiotic are administered to a subject simultaneously. In some embodiments, the peptide multimer and the carbapenem antibiotic are administered to a subject sequentially. For example, a peptide multimer can be administered to a subject, after which a carbapenem antibiotic can be administered to the subject. Alternatively, a carbapenem antibiotic can be administered to a subject, after which a peptide multimer can be administered to the subject.

Compositions and methods provided herein can be practiced by delivering the peptide multimer and/or carbapenem antibiotic of the present disclosure using a means for delivery e.g., any suitable carrier. The dose of peptide multimer and/or carbapenem antibiotic, mode of administration and use of suitable carrier will depend upon the intended patient or subject and the targeted bacteria. The formulations, both for human medical use and veterinary use, of peptide multimers and/or carbapenem antibiotics according to the present disclosure typically include such peptide multimers and/or carbapenem antibiotics in association with a pharmaceutically acceptable carrier.

The carrier(s) should be “acceptable” in the sense of being compatible with the peptide multimer and/or carbapenem antibiotic of the present disclosure and not deleterious to the recipient. Pharmaceutically acceptable carriers, in this regard, are intended to include any and all solvents, dispersion media, coatings, absorption delaying agents, and the like, compatible with pharmaceutical administration. Supplementary active compounds (identified or designed according to the disclosure and/or known in the art) also can be incorporated into the compositions. In some embodiments, formulations are prepared by bringing the peptide multimer and/or carbapenem antibiotic into association with a liquid carrier or a finely divided solid carrier or both, and then, if necessary, shaping the product into the desired formulation.

A pharmaceutical composition should be formulated to be compatible with its intended route of administration. Solutions or suspensions can include the following components: a sterile diluent such as water, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide.

Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions that are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations suitable for oral administration can be in the form of: discrete units such as capsules, gelatin capsules, sachets, tablets, troches, or lozenges, each containing a predetermined amount of the peptide multimer and/or carbapenem antibiotic of the present disclosure; a powder or granular composition; a solution or a suspension in an aqueous liquid or non-aqueous liquid; or an oil-in-water emulsion or a water-in-oil emulsion. The peptide multimer and/or carbapenem antibiotic of the present disclosure can also be administered in the form of a bolus, electuary or paste. A tablet can be made by compressing or molding the peptide multimer and/or carbapenem antibiotic of the present disclosure optionally with one or more accessory ingredients. Compressed tablets can be prepared by compressing, in a suitable machine, the peptide multimer and/or carbapenem antibiotic of the present disclosure in a free-flowing form such as a powder or granules, optionally mixed by a binder, lubricant, inert diluent, surface active or dispersing agent. Molded tablets can be made by molding, in a suitable machine, a mixture of the powdered peptide multimer and/or carbapenem antibiotic of the present disclosure and suitable carrier moistened with an inert liquid diluent.

Oral compositions generally include an inert diluent or an edible carrier. For the purpose of oral therapeutic administration, the peptide multimer and/or carbapenem antibiotic of the present disclosure can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the peptide multimer and/or carbapenem antibiotic of the present disclosure in the required amount in an appropriate solvent with one or a combination of ingredients enumerated above, as required, followed by filter sterilization. Generally, dispersions are prepared by incorporating the peptide multimer and/or carbapenem antibiotic of the present disclosure into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the peptide multimer and/or carbapenem antibiotic of the present disclosure that can be in microcrystalline form, for example, in the form of an aqueous microcrystalline suspension. Liposomal formulations or biodegradable polymer systems can also be used to present the peptide multimer and/or carbapenem antibiotic of the present disclosure for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the peptide multimer and/or carbapenem antibiotic of the present disclosure with a dermatologically acceptable carrier such as a lotion, cream, ointment or soap. Useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect can be achieved either by choice of a valve having the desired spray characteristics (i.e., being capable of producing a spray having the desired particle size) or by incorporating the peptide multimer and/or carbapenem antibiotic of the present disclosure as a suspended powder in controlled particle size. For administration by inhalation, the peptide multimer and/or carbapenem antibiotic of the present disclosure also can be delivered in the form of an aerosol spray from pressured container or dispenser, which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants can include, for example, for transmucosal administration, detergents and bile salts. Transmucosal administration can be accomplished through the use of nasal sprays or suppositories. For transdermal administration, the peptide multimer and/or carbapenem antibiotic of the present disclosure typically are formulated into ointments, salves, gels, or creams.

The peptide multimer and/or carbapenem antibiotic can be prepared with carriers that will protect the peptide multimer and/or carbapenem antibiotic against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Liposomal suspensions can also be used as pharmaceutically acceptable carriers.

Oral or parenteral compositions can be formulated in dosage unit form for ease of administration and uniformity of dosage. Dosage unit form refers to physically discrete units suited as unitary dosages for the subject to be treated; each unit containing a predetermined quantity of the peptide multimer and/or carbapenem antibiotic calculated to produce the desired therapeutic effect in association with the required pharmaceutical carrier. The specification for the dosage unit forms of the disclosure are dictated by and directly dependent on the unique characteristics of the peptide multimer and/or carbapenem antibiotic and the therapeutic effect to be achieved, and the limitations inherent in the art of compounding such a peptide multimer and/or carbapenem antibiotic for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired, the composition can include the drug dispersed in a fibrinogen-thrombin composition or other bioadhesive. The peptide multimer and/or carbapenem antibiotic then can be painted, sprayed or otherwise applied to the desired tissue surface. Alternatively, the peptide multimer and/or carbapenem antibiotic can be formulated for parenteral or oral administration to humans or other mammals, for example, in effective amounts, e.g., amounts that provide appropriate concentrations of the peptide multimer and/or carbapenem antibiotic to target tissue for a time sufficient to induce the desired effect.

Generally, an effective amount of dosage of peptide multimer and/or carbapenem antibiotic of the present disclosure will be in the range of from about 0.1 mg/kg to about 100 mg/kg of body weight/day, for example, from about 1.0 mg/kg to about 50 mg/kg of body weight/day. In some embodiments, the dosage of active compound is in the range of from about 0.1 mg/kg to about 1.0 mg/kg of body weight/day; from about 0.1 mg/kg to about 5 mg/kg of body weight/day; from about 0.1 mg/kg to about 10 mg/kg of body weight/day; from about 0.1 mg/kg to about 25 mg/kg of body weight/day; from about 0.1 mg/kg to about 50 mg/kg of body weight/day; from about 1.0 mg/kg to about 5.0 mg/kg of body weight/day; from about 1.0 mg/kg to about 10 mg/kg of body weight/day; from about 1.0 mg/kg to about 20 mg/kg of body weight/day; from about 1.0 mg/kg to about 25 mg/kg of body weight/day; from about 1.0 mg/kg to about 40 mg/kg of body weight/day; from about 1.0 mg/kg to about 100 mg/kg of body weight/day; from about 10 mg/kg to about 100 mg/kg of body weight/day; from about 25 mg/kg to about 100 mg/kg of body weight/day; from about 50 mg/kg to about 100 mg/kg of body weight/day; from about 5.0 mg/kg to about 50 mg/kg of body weight/day; from about 10 mg/kg to about 50 mg/kg of body weight/day; or from about 25 mg/kg to about 50 mg/kg of body weight/day.

The amount administered will also likely depend on such variables as the type of surgery or invasive medical procedure, the overall health status of the patient, the relative biological efficacy of the peptide multimer and/or carbapenem antibiotic of the present disclosure delivered, the formulation of the peptide multimer and/or carbapenem antibiotic, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

Nonlimiting doses of the peptide multimer and/or carbapenem antibiotic of the present disclosure comprise from about 0.1 mg to about 1500 mg per dose. For example, a dose of active compound can range from about 0.1 mg to about 1250 mg; about 0.1 mg to about 1000 mg; about 0.1 mg to about 800 mg; about 0.1 mg to about 500 mg; about 0.1 mg to about 250 mg; about 0.1 mg to about 100 mg; about 0.1 mg to about 50 mg; about 0.1 mg to about 25 mg; about 0.1 mg to about 20 mg; about 0.1 mg to about 10 mg; about 0.1 mg to about 5 mg; about 0.1 mg to about 1 mg; about 0.1 mg to about 0.5 mg; about 0.5 mg to about 1500 mg; about 1 mg to about 1500 mg; about 2.5 mg to about 1500 mg; about 5 mg to about 1500 mg; about 10 mg to about 1500 mg; about 50 mg to about 1500 mg; about 100 mg to about 1500 mg; about 250 mg to about 1500 mg; about 500 mg to about 1500 mg; about 750 mg to about 1500 mg; about 1000 mg to about 1500 mg; about 1250 mg to about 1500 mg; about 0.25 mg to about 2.5 mg; about 0.5 mg to about 5 mg; about 1 mg to about 10 mg; about 5 to about 20 mg; about 10 mg to about 50 mg; about 25 mg to about 75 mg; about 20 mg to about 100 mg; about 50 mg to about 200 mg; about 100 mg to about 500 mg; about 250 mg to about 750 mg; about 200 mg to about 800 mg; about 500 mg to about 1000 mg; or about 750 mg to about 1250 mg. In some embodiments, any of the variety of peptide multimers and/or carbapenem antibiotics described herein are administered at one time in a single dose (e.g., any of the variety of dosage amounts described herein). In some embodiments, any of the variety of peptide multimers and/or carbapenem antibiotics described herein are administered over time in two or more doses. For example, a peptide multimer and/or a carbapenem antibiotic can be administered in two or more doses over the course of 4 hours, 6 hours, 8 hours, 10 hours, 12 hours, 14 hours, 16 hours, 18 hours, 20 hours, 22 hours, 24 hours, or longer, such that the total dose is any of the variety of dosage amounts described herein). In embodiments in which a peptide multimer and/or a carbapenem antibiotic is administered over time in two or more doses, each of the two or more doses can comprise the same amount of the peptide multimer and/or the carbapenem antibiotic, or different amounts.

As is understood by one of ordinary skill in the art, generally, when dosages are described for a pharmaceutical active, the dosage is given on the basis of the parent or active moiety. Therefore, if a salt, hydrate, or another form of the parent or active moiety is used, a corresponding adjustment in the weight of the compound is made, although the dose is still referred to on the basis of the parent or active moiety delivered. As a nonlimiting example, if the parent or active moiety of interest is a monocarboxylic acid having a molecular weight of 250, and if the monosodium salt of the acid is desired to be delivered to be delivered at the same dosage, then an adjustment is made recognizing that the monosodium salt would have a molecular weight of approximately 272 (i.e., minus 1H or 1.008 atomic mass units and plus 1 Na or 22.99 atomic mass units). Therefore, a 250 mg dosage of the parent or active compound would correspond to about 272 mg of the monosodium salt, which would also deliver 250 mg of the parent or active compound. Put another way, about 272 mg of the monosodium salt would be equivalent to a 250 mg dosage of the parent or active compound.

Methods of Eliminating and/or Inhibiting Bacteria

Provided herein are methods of eliminating (e.g., killing) bacteria, as well as methods inhibiting or preventing bacterial growth with a peptide multimer and a carbapenem antibiotic (e.g., any of the variety of peptide multimers and carbapenem antibiotics described herein), wherein such elimination or inhibition is done outside the context of a medical treatment.

In some embodiments, peptide multimers and carbapenem antibiotics provided herein can be used to remove, reduce, or prevent growth of planktonic bacterial forms. In some embodiments, peptide multimers and carbapenem antibiotics provided herein can be used to remove, reduce, or prevent formation of biofilms. The formation of biofilms is a significant problem that is implicated in a variety of settings both the medical field and the non-medical field. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids, and proteins) and nutrients allows for enhanced microbial cross-talk and increased virulence. As biofilms may develop in any supporting environment, a method or composition that can remove, reduce, or prevent formation of biofilms is useful. In some embodiments, a peptide multimer and a carbapenem antibiotic exhibit improved (e.g., synergistic) antibacterial activity against biofilms as compared to either the peptide multimer or the carbapenem antibiotic alone. In some embodiments, any of the variety of peptide multimers and carbapenem antibiotics provided herein inhibit growth of bacteria in a biofilm (e.g., any of the variety of bacterial genii or species provided herein) by at least 50%, at least 55%, at least 60%, at least 65%, at least 70%, at least 75%, at least 80%, at least 85%, at least 90%, at least 95%, at least 96%, at least 97%, at least 98%, at least 99%, or more. In some embodiments, any of the variety of peptide multimers and carbapenem antibiotics provided herein inhibit growth of bacteria in a biofilm (e.g., any of the variety of bacterial genii or species provided herein) to a greater degree than either the peptide multimer or the carbapenem antibiotic alone. In some embodiments, any of the variety of peptide multimers and carbapenem antibiotics provided herein inhibit growth of bacteria in a biofilm (e.g., any of the variety of bacterial genii or species provided herein) by a factor of at least 2-fold, at least 3-fold, at least 4-fold, at least 5-fold, at least 6-fold, at least 7-fold, at least 8-fold, at least 9-fold, at least 10-fold, or more as compared to the peptide multimer or the carbapenem antibiotic alone. In some embodiments, any of the variety of peptide multimers and carbapenem antibiotics provided herein eliminate (e.g., kill) at least 50%, at least 55%, at least 60%, at least 65%, at least 70%, at least 75%, at least 80%, at least 85%, at least 90%, at least 95%, at least 96%, at least 97%, at least 98%, at least 99%, or more bacteria in a biofilm (e.g., bacteria of any of the variety of bacterial genii or species provided herein). In some embodiments, any of the variety of peptide multimers and carbapenem antibiotics provided herein eliminate (e.g., kill) bacteria in a biofilm (e.g., any of the variety of bacterial genii or species provided herein to a greater degree than either the peptide multimer or the carbapenem antibiotic alone. In some embodiments, any of the variety of peptide multimers and carbapenem antibiotics provided herein eliminate (e.g., kill) bacteria in a biofilm (e.g., bacteria of any of the variety of bacterial genii or species provided herein) by a factor of at least 2-fold, at least 3-fold, at least 4-fold, at least 5-fold, at least 6-fold, at least 7-fold, at least 8-fold, at least 9-fold, at least 10-fold, or more as compared to the peptide multimer or the carbapenem antibiotic alone. In some embodiments, peptide multimers and carbapenem antibiotics provided herein have antibacterial activity against bacteria in a biofilm (e.g., synergistic antibacterial activity) as compared to the peptide multimer or the carbapenem antibiotic alone, regardless of whether the bacteria of the biofilm produce beta-lactamase enzymes. In some embodiments, peptide multimers and carbapenem antibiotics provided that remove, reduce, or prevent formation of biofilms are applied to the biofilm simultaneously. In some embodiments, peptide multimers and carbapenem antibiotics provided that remove, reduce, or prevent formation of biofilms are applied to the biofilm sequentially. For example, a peptide multimer can be applied to a biofilm, after which a carbapenem antibiotic can be applied to the biofilm. Alternatively, a carbapenem antibiotic can be applied to a biofilm, after which a peptide multimer can be applied to the biofilm.

In some embodiments, provided herein are methods of removing a biofilm using any of the variety of peptide multimers and carbapenem antibiotics described herein. In some embodiments, a biofilm can occur on a surface. The terms “surface” or “surfaces” used herein, refer to any surface whether medical or industrial, that provides an interface between a fluid, such as a liquid or air, and a solid. The interface between fluid and solid can be intermittent, and can be caused by flowing or stagnant fluid, aerosols, or other means for air-borne fluid exposure. A surface refers, in some examples, to a plane whose mechanical structure is compatible with the adherence of bacteria or fungi. In the context of peptide multimers and carbapenem antibiotics and methods described herein, the terminology “surface” encompasses the inner and outer aspects of various instruments and devices, both disposable and non-disposable, medical and nonmedical.

Peptide multimers and carbapenem antibiotics described herein can be used to remove, reduce, or prevent formation of biofilms in non-medical contexts (e.g., biofilms present on non-medical surfaces) including, without limitation, the hulls of a ship, dockyards, food processors, mixers, machines, containers, water tanks, water filtration systems, commercial or residential swimming pools, purification systems, preservatives in food industries, personal care products such as shampoo, cream, moisturizer, hand sanitizer, soaps and the like.

Peptide multimers and carbapenem antibiotics described herein can be used to remove, reduce, or prevent formation of biofilms in medical contexts (e.g., biofilms present on medical surfaces). Medical surfaces can include the inner and outer aspects of various instruments and devices, whether disposable or intended for repeated uses. Non-limiting examples include the entire spectrum of articles adapted for medical use, including scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopaedic pins, plates and implants; catheters and other tubes (including urological and biliary tubes, endotracheal tubes, peripherally insertable central venous catheters, dialysis catheters, long term tunnelled central venous catheters, peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venpus shunts); prostheses (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, dental implants, filings, dentures and the like. Surfaces found in the medical environment also include the inner and outer aspects of pieces of medical equipment, medical gear worn or carried by personnel in the health care setting. Such surfaces can include counter tops and fixtures in areas used for medical procedures or for preparing medical apparatus, tubes and canisters used in respiratory treatments, including the administration of oxygen, of solubilised drugs in nebulisers and of aesthetic agents. Also included are those surfaces intended as biological barriers to infectious organisms in medical settings, such as gloves, aprons and face-shields. Commonly used materials for biological barriers may be latex-based or non- latex based. A non-limiting example for a non-latex based biological barrier material includes vinyl. Other such surfaces can include handles and cables for medical or dental equipment not intended to be sterile. Additionally, such surfaces can include those non-sterile external surfaces of tubes and other apparatus found in areas where blood or body fluids or other hazardous biomaterials are commonly encountered. As one non-limiting example, the biofilm may be present on catheters and medical implants. Other examples will be known to a person of ordinary skill in the art.

In some embodiments, a single carbapenem antibiotic (e.g., any of the variety of carbapenem antibiotics disclosed herein or known in the art) and a single peptide multimer (e.g., any of the variety of peptide multimers disclosed herein or known in the art) are used to eliminate (e.g., kill) bacteria or to inhibit or prevent bacterial growth. In embodiments, a single carbapenem antibiotic and two or more (e.g., 2, 3, 4, 5, 6, 7, 8, 9, 10, or more) peptide multimers are used to eliminate (e.g., kill) bacteria or to inhibit or prevent bacterial growth. In some embodiments two or more (e.g., 2, 3, 4, 5, 6, 7, 8, 9, 10, or more) carbapenem antibiotics and a single peptide multimer are used to eliminate (e.g., kill) bacteria or to inhibit or prevent bacterial growth. In some embodiments, two or more (e.g., 2, 3, 4, 5, 6, 7, 8, 9, 10, or more) carbapenem antibiotics and two or more (e.g., 2, 3, 4, 5, 6, 7, 8, 9, 10, or more) peptide multimers are used to eliminate (e.g., kill) bacteria or to inhibit or prevent bacterial growth.

In some embodiments, a peptide dimer and a carbapenem antibiotic (e.g., any of the variety of carbapenem antibiotics described herein or known in the art) are used to eliminate (e.g., kill) bacteria or to inhibit or prevent bacterial growth. Non limiting examples of peptide dimers and carbapenem antibiotics that can be used to eliminate (e.g., kill) bacteria or to inhibit or prevent bacterial growth include: B2088 (e.g., lipidated or non-lipidated) and meropenem, B2088 (e.g., lipidated or non-lipidated) and imipenem, B2088 (e.g., lipidated or non-lipidated) and doripenem, B2088 (e.g., lipidated or non-lipidated) and eratapenem, B2088 (e.g., lipidated or non-lipidated) and panipenem, B2088 (e.g., lipidated or non-lipidated) and biapenem, B2088 (e.g., lipidated or non-lipidated) and tebipenem, B2088 (e.g., lipidated or non-lipidated) and razupenem, B2088 (e.g., lipidated or non-lipidated) and lenapenem, B2088 (e.g., lipidated or non-lipidated) and tomopenem, and B2088 (e.g., lipidated or non-lipidated) and tomopenem.

Other non-limiting examples of peptide dimers and carbapenem antibiotics that can be used to eliminate (e.g., kill) bacteria or to inhibit or prevent bacterial growth include: a peptide dimer (e.g., lipidated or non-lipidated) having at least one monomer subunit (e.g., two monomer subunits) of any one of SEQ ID NO: 20-46 and meropenem, a peptide dimer (e.g., lipidated or non-lipidated) having at least one monomer subunit (e.g., two monomer subunits) of any one of SEQ ID NO: 20-46 and imipenem, a peptide dimer (e.g., lipidated or non-lipidated) having at least one monomer subunit (e.g., two monomer subunits) of any one of SEQ ID NO: 20-46 and doripenem, a peptide dimer (e.g., lipidated or non-lipidated) having at least one monomer subunit (e.g., two monomer subunits) of any one of SEQ ID NO: 20-46 and eratapenem, a peptide dimer (e.g., lipidated or non-lipidated) having at least one monomer subunit (e.g., two monomer subunits) of any one of SEQ ID NO: 20-46 and panipenem, a peptide dimer (e.g., lipidated or non-lipidated) having at least one monomer subunit (e.g., two monomer subunits) of any one of SEQ ID NO: 20-46 and biapenem, a peptide dimer (e.g., lipidated or non-lipidated) having at least one monomer subunit (e.g., two monomer subunits) of any one of SEQ ID NO: 20-46 and tebipenem, a peptide dimer (e.g., lipidated or non-lipidated) having at least one monomer subunit (e.g., two monomer subunits) of any one of SEQ ID NO: 20-46 and razupenem, a peptide dimer (e.g., lipidated or non-lipidated) having at least one monomer subunit (e.g., two monomer subunits) of any one of SEQ ID NO: 20-46 and lenapenem, a peptide dimer (e.g., lipidated or non-lipidated) having at least one monomer subunit (e.g., two monomer subunits) of any one of SEQ ID NO: 20-46 and tomopenem, and a peptide dimer (e.g., lipidated or non-lipidated) having at least one monomer subunit (e.g., two monomer subunits) of any one of SEQ ID NO: 20-46 and tomopenem. In some embodiments, the two peptide monomer subunits of the peptide dimer are the same. In some embodiments, the two peptide monomer subunits of the peptide dimer are different.

In some embodiments, a peptide tetramer and a carbapenem antibiotic (e.g., any of the variety of carbapenem antibiotics described herein or known in the art) are used to eliminate (e.g., kill) bacteria or to inhibit or prevent bacterial growth. Non-limiting examples of peptide tetramers and carbapenem antibiotics that can be used to eliminate (e.g., kill) bacteria or to inhibit or prevent bacterial growth include: a peptide tetramer (e.g., lipidated or non-lipidated) having at least one monomer subunit (e.g., one, two, three, or four monomer subunits) of any one of SEQ ID NO: 20-46 and meropenem, a peptide tetramer (e.g., lipidated or non-lipidated) having at least one monomer subunit (e.g., one, two, three, or four monomer subunits) of any one of SEQ ID NO: 20-46 and imipenem, a peptide tetramer (e.g., lipidated or non-lipidated) having at least one monomer subunit (e.g., one, two, three, or four monomer subunits) of any one of SEQ ID NO: 20-46 and doripenem, a peptide tetramer (e.g., lipidated or non-lipidated) having at least one monomer subunit (e.g., one, two, three, or four monomer subunits) of any one of SEQ ID NO: 20-46 and eratapenem, a peptide tetramer (e.g., lipidated or non-lipidated) having at least one monomer subunit (e.g., one, two, three, or four monomer subunits) of any one of SEQ ID NO: 20-46 and panipenem, a peptide tetramer (e.g., lipidated or non-lipidated) having at least one monomer subunit (e.g., one, two, three, or four monomer subunits) of any one of SEQ ID NO: 20-46 and biapenem, a peptide tetramer (e.g., lipidated or non-lipidated) having at least one monomer subunit (e.g., one, two, three, or four monomer subunits) of any one of SEQ ID NO: 20-46 and tebipenem, a peptide tetramer (e.g., lipidated or non-lipidated) having at least one monomer subunit (e.g., one, two, three, or four monomer subunits) of any one of SEQ ID NO: 20-46 and razupenem, a peptide tetramer (e.g., lipidated or non-lipidated) having at least one monomer subunit (e.g., one, two, three, or four monomer subunits) of any one of SEQ ID NO: 20-46 and lenapenem, a peptide tetramer (e.g., lipidated or non-lipidated) having at least one monomer subunit (e.g., one, two, three, or four monomer subunits) of any one of SEQ ID NO: 20-46 and tomopenem, and a peptide tetramer (e.g., lipidated or non-lipidated) having at least one monomer subunit (e.g., one, two, three, or four monomer subunits) of any one of SEQ ID NO: 20-46 and tomopenem. In some embodiments, at least two (e.g., 2, 3 or 4) peptide monomer subunits of the peptide tetramer are the same. In some embodiments, at least two (e.g., 2, 3 or 4) peptide monomer subunits of the peptide tetramer are different.

### Antibacterial Compositions

Also provided herein are antibacterial compositions that include a peptide multimer and a carbapenem antibiotic. In some embodiments, antibacterial compositions that include a peptide multimer and a carbapenem antibiotic exhibit improved (e.g., synergistic) antibacterial activity as compared to a reference composition that lacks either the peptide multimer or the carbapenem. For example, antibacterial compositions that include a peptide multimer and a carbapenem antibiotic can exhibit improved (e.g., synergistic) antibacterial activity as compared to the peptide multimer or the carbapenem antibiotic alone (e.g., the antibacterial composition exhibits a more than additive effect when compared to either the peptide multimer or the carbapenem antibiotic alone).

In some embodiments, any of the variety of antibacterial compositions provided herein that include a peptide multimer and a carbapenem antibiotic inhibit growth of a bacterium (e.g., a bacterium of any of the variety of bacterial genii or species provided herein) by at least 50%, at least 55%, at least 60%, at least 65%, at least 70%, at least 75%, at least 80%, at least 85%, at least 90%, at least 95%, at least 96%, at least 97%, at least 98%, at least 99%, or more. In some embodiments, any of the variety of antibacterial compositions provided herein that include a peptide multimer and a carbapenem antibiotic inhibit growth of a bacterium as compared to the peptide multimer or the carbapenem antibiotic alone. In some embodiments, any of the variety of antibacterial compositions provided herein that include a peptide multimer and a carbapenem antibiotic inhibit growth of a bacterium (e.g., a bacterium of any of the variety of bacterial genii or species provided herein) by a factor of at least 2-fold, at least 3-fold, at least 4-fold, at least 5-fold, at least 6-fold, at least 7-fold, at least 8-fold, at least 9-fold, at least 10-fold, or more as compared to either the peptide multimer or the carbapenem antibiotic alone. In some embodiments, any of the variety of antibacterial compositions provided herein that include a peptide multimer and a carbapenem antibiotic eliminate (e.g., kill) at least 50%, at least 55%, at least 60%, at least 65%, at least 70%, at least 75%, at least 80%, at least 85%, at least 90%, at least 95%, at least 96%, at least 97%, at least 98%, at least 99%, or more bacteria (e.g., bacteria of any of the variety of bacterial genii or species provided herein). In some embodiments, any of the variety of antibacterial compositions provided herein that include a peptide multimer and a carbapenem antibiotic eliminate (e.g., kill) bacteria more effectively as compared to either the peptide multimer or the carbapenem antibiotic alone. In some embodiments, any of the variety of antibacterial compositions provided herein that include a peptide multimer and a carbapenem antibiotic eliminate (e.g., kill) bacteria (e.g., bacteria of any of the variety of bacterial genii or species provided herein) by a factor of at least 2-fold, at least 3-fold, at least 4-fold, at least 5-fold, at least 6-fold, at least 7-fold, at least 8-fold, at least 9-fold, at least 10-fold, or more as compared to either the peptide multimer or the carbapenem antibiotic alone. In some embodiments, antibacterial compositions provided herein that include a peptide multimer and a carbapenem antibiotic have antibacterial activity against a given bacterium (e.g., improved antibacterial activity, e.g., synergistic antibacterial activity), regardless of whether the bacterium produces beta-lactamase enzymes.

In some embodiments, any of the variety of antibacterial compositions provided herein that include a peptide multimer and a carbapenem antibiotic include a peptide multimer present in an amount that is lower than a corresponding amount of the peptide multimer required to achieve similar antibacterial effects in the absence of the carbapenem antibiotic. In some embodiments, any of the variety of antibacterial compositions provided herein that include a peptide multimer and a carbapenem antibiotic include a carbapenem antibiotic present in an amount that is lower than a corresponding amount of the carbapenem antibiotic required to achieve similar antibacterial effects in the absence of the peptide multimer. Nonlimiting amounts of peptide multimers and/or carbapenem antibiotics that can be included in antibacterial compositions provided herein can be from about 0.1 mg to about 1500 mg per dose. For example, an antibacterial composition can include a peptide multimer and/or a carbapenem antibiotic that is present in an amount from about 0.1 mg to about 1250 mg; about 0.1 mg to about 1000 mg; about 0.1 mg to about 800 mg; about 0.1 mg to about 500 mg; about 0.1 mg to about 250 mg; about 0.1 mg to about 100 mg; about 0.1 mg to about 50 mg; about 0.1 mg to about 25 mg; about 0.1 mg to about 20 mg; about 0.1 mg to about 10 mg; about 0.1 mg to about 5 mg; about 0.1 mg to about 1 mg; about 0.1 mg to about 0.5 mg; about 0.5 mg to about 1500 mg; about 1 mg to about 1500 mg; about 2.5 mg to about 1500 mg; about 5 mg to about 1500 mg; about 10 mg to about 1500 mg; about 50 mg to about 1500 mg; about 100 mg to about 1500 mg; about 250 mg to about 1500 mg; about 500 mg to about 1500 mg; about 750 mg to about 1500 mg; about 1000 mg to about 1500 mg; about 1250 mg to about 1500 mg; about 0.25 mg to about 2.5 mg; about 0.5 mg to about 5 mg; about 1 mg to about 10 mg; about 5 to about 20 mg; about 10 mg to about 50 mg; about 25 mg to about 75 mg; about 20 mg to about 100 mg; about 50 mg to about 200 mg; about 100 mg to about 500 mg; about 250 mg to about 750 mg; about 200 mg to about 800 mg; about 500 mg to about 1000 mg; or about 750 mg to about 1250 mg.

Any of a variety of peptide multimers having antibacterial activity can be included in the antibacterial compositions described herein. In some embodiments, a peptide multimer includes a non-linear peptide multimer (e.g., a branched peptide multimer). Non-limiting examples of branched peptide multimers include any peptide multimers described in U.S. Pat. Nos. 8,809,262 or 9,220,264, or U.S. Patent Application Publication Numbers 2015/0225454 or 2015/0231199, each of which is incorporated herein by reference in its entirety.

In some embodiments, a peptide multimer is branched at a terminal or penultimate amino acid residue (e.g., at a terminal or penultimate lysine residue). In some embodiments, any of the variety of antibacterial compositions provided herein include a single peptide multimer (e.g., any of the variety of peptide multimers disclosed herein). In embodiments of antibacterial compositions that include a single peptide multimer, the antibacterial composition can include a single or two or more (e.g., 2, 3, 4, 5, 6, 7, 8, 9, 10, or more) carbapenem antibiotics (e.g., any of the variety of carbapenem antibiotics disclosed herein). In some embodiments, any of the variety of antibacterial compositions provided herein include two or more (e.g., 2, 3, 4, 5, 6, 7, 8, 9, 10, or more) peptide multimers (e.g., any of the variety of peptide multimers disclosed herein). In embodiments of antibacterial compositions that include two or more peptide multimers, the antibacterial composition can include a single or two or more (e.g., 2, 3, 4, 5, 6, 7, 8, 9, 10, or more) carbapenem antibiotics (e.g., any of the variety of carbapenem antibiotics disclosed herein).

In some embodiments, antibacterial compositions provided herein include a peptide multimer having monomeric subunits that include a portion of a defensin peptide. For example, an antibacterial composition can include a peptide multimer having one or more monomeric subunits that include sequences present in a human defensin peptide such as, without limitation, hBD3 (GIINTLQKYYCRVRGGRCAVLSCLPKEEQIGKCSTRGRKCCRRKK, SEQ ID NO: 1). In some embodiments, a peptide multimer includes one or more monomeric subunits that include sequences that differ from sequences present in a human defensin peptide by one or more amino acid residues.

In some embodiments, antibacterial compositions provided herein that include a peptide multimer and a carbapenem antibiotic (e.g., any of the carbapenem antibiotics described herein) include a peptide multimer of the formula (U)nBmZj, wherein U is a peptide monomer comprising RGRKVVRR (SEQ ID NO: 2) or a variant thereof comprising at least one amino acid substitution, at least one amino acid deletion, a rearrangement of at least one peptide monomer compared to the initial peptide monomer and/or at least one non-proteogenic amino acid modification in at least one amino acid, wherein n≥2, m≥1, and j≥0 wherein each B comprises at least one amino acid having at least two amine groups, wherein Z comprises any amino acid, and wherein the multimer is branched at the terminal BmZj residues. In some embodiments, each of the U peptide monomers in the peptide multimer is the same sequence. In some embodiments, at least one of the U peptide monomers in the peptide multimer is a different sequence than another U peptide monomer of the peptide multimer.

In some embodiments, antibacterial compositions provided herein that include a peptide multimer and a carbapenem antibiotic (e.g., any of the carbapenem antibiotics described herein) include a peptide multimer of Formula I

[(R)ₐ(X¹)_{b}(X²)_{c}(X³)ₐ(X⁴)_{b}]ₙ

wherein X¹, X² and X⁴ are independently of each other selected from the group consisting of lysine (K), arginine (R), glycine (G) and alanine (A); and X³ is lysine (K), arginine (R), leucine (L), valine (V), isoleucine (I), glycine (G) or alanine (A). In some embodiments, a and b are independently selected to be an integer from 1 to 10. In some embodiments, c is an integer selected from 0 to 5. In some embodiments, n is at least one. In some embodiments, a and b may be independently selected from an integer from 1 to 10. In some embodiments, a and b may be the same or different from one another. In some embodiments, a may be 1, 2, 3, 4, 5, 6, 7, 8, 9 or 10. In some embodiments, b may be 1, 2, 3, 4, 5, 6, 7, 8, 9 or 10. As non-limiting examples, a may be 1 and b may be 1; a may be 1 and b may be 2; a may be 1 and b may be 3; a may be 1 and b may be 4; a may be 1 and b may be 5; a may be 1 and b may be 6; a may be 1 and b may be 7; a may be 1 and b may be 8; a may be 1 and b may be 9; a may be 1 and b may be 10; a may be 2 and b may be 1; a may be 3 and b may be 1; a may be 4 and b may be 1; a may be 5 and b may be 1; a may be 6 and b may be 1; a may be 7 and b may be 1; a may be 8 and b may be 1; a may be 9 and b may be 1; a may be 10 and b may be 1; a may be 2 and b may be 2; a may be 2 and b may be 3; a may be 2 and b may be 4; or any combination thereof.

In some embodiments, antibacterial compositions provided herein that include a peptide multimer and a carbapenem antibiotic (e.g., any of the carbapenem antibiotics described herein) include a peptide multimer that does not include hydrophobic amino acids. In some embodiments, such a peptide multimer exhibits enhanced antimicrobial activity as compared to a peptide multimer that includes hydrophobic amino acids. For example, X¹, X², X³ and X⁴ of Formula I (SEQ ID NO: 1) may be independently of each other selected from the group consisting of non-hydrophobic amino acids. In some embodiments, X¹, X², X³and X⁴ may be the same or different from one another. In some embodiments, X¹, X², X³and X⁴ may not amino acids other than hydrophobic amino acids. In some embodiments, X¹, X², X³ and X⁴ may be amino acids other than valine (V), isoleucine (I), leucine (L), methionine (M), phenylalanine (F), tyrosine (Y), or tryptophan (W). In some embodiments, X¹, X², X³ and X⁴ may be neutral amino acids. In some embodiments, X¹, X², X³ and X⁴ may be cationic amino acids. In some embodiments, X¹, X², X³ and X⁴ may be independently of each other an amino acid including, but not limited to arginine (R), histidine (H), lysine (K), aspartic acid (D), glutamic acid (E), serine (S), threonine (T), asparagine (N), glutamine (Q), cysteine (C), glycine (G), proline (P) or alanine (A). In some embodiments, X^{l}, X², X³ and X⁴ may be lysine (K), arginine (R), glycine (G) or alanine (A). In some embodiments, X¹ may be lysine (K), arginine (R), glycine (G) or alanine (A). In some embodiments, X may be lysine (K), arginine (R), glycine (G) or alanine (A). In some embodiments, X³ may be lysine (K), arginine (R), glycine (G) or alanine (A). In some embodiments, X⁴ may be lysine (K), arginine (R), glycine (G) or alanine (A). X¹, X², X³ and X⁴ may be any combination of the aforementioned amino acids. For example, X¹ may be a lysine (K), X² may be a glycine (G) or alanine (A), X³ may be an arginine (R) and X⁴ may be a lysine (K). As an additional example, X^{l} may be a glycine (G) or alanine (A), X² may be a lysine (K), X³ may be a glycine (G) or alanine (A), and X⁴ may be an arginine (R).

In some embodiments of antibacterial compositions having a carbapenem antibiotic (e.g., any of the carbapenem antibiotics described herein) and a peptide multimer of Formula 1, when n is at least 2, each of the peptide monomers is linked to at least two lysine (K) residues. As used herein, “linked” refers to two sequences of a peptide being coupled or connected to one other in a manner that permits each peptide branch to move freely of each other. In some embodiments, “linked” peptide monomer sequences are immediately adjacent to one another. In some embodiments, a plurality of monomers of the peptide as described herein is linked by lysine (K) residue(s) via covalent bonds. In some embodiments, the lysine (K) linkage is at the C-terminal end of the peptide multimer. The term “C- terminal end” is used herein in accordance to its definition as commonly known in the art, that is, can be used interchangeably with any of the following terminologies such as the carboxyl-terminus, carboxy-terminus, C-terminal tail, C-terminus or COOH-terminus, which refer to the end of an amino acid chain, terminated by a free carboxyl group (—COOH).

In some embodiments, antibacterial compositions provided herein that include a peptide multimer and a carbapenem antibiotic (e.g., any of the carbapenem antibiotics described herein) include a carbapenem antibiotic and a peptide multimer of Formula I having peptide monomer subunits that are linked by a lysine (K) linkage, wherein n is 2 (e.g., the peptide is a dimer). In such embodiments, the peptide multimer may have the structure:

[(R)ₐ(X¹)_{b}(X²)_{c}(X³)ₐ(X⁴)_{b}]₂KK or

[(R)ₐ(X¹)_{b}(X²)_{c}(X³)ₐ(X⁴)_{b}]—K—K—[(X⁴)_{b}(X³)ₐ(X²)_{c}(X¹)_{b}(R)ₐ].

In some embodiments, antibacterial compositions provided herein that include a peptide multimer and a carbapenem antibiotic (e.g., any of the carbapenem antibiotics described herein) include a carbapenem antibiotic and a peptide multimer of Formula I having peptide monomer subunits that are linked by a lysine (K) linkage, wherein n is 3 (e.g., the peptide is a trimer). In such embodiments, the peptide multimer may have the structure:

[(R)ₐ(X¹)_{b}(X²)_{c}(X³)ₐ(X⁴)_{b}]₃K₂K

In some embodiments, antibacterial compositions provided herein that include a peptide multimer and a carbapenem antibiotic (e.g., any of the carbapenem antibiotics described herein) include a carbapenem antibiotic and a peptide multimer of Formula I having peptide monomer subunits that are linked by a lysine (K) linkage, wherein n is 4 (e.g., the peptide is a tetramer). In such embodiments, the peptide multimer may have the structure:

[(R)ₐ(X¹)_{b}X²)_{c}(X³)ₐ(X⁴)_{b}]₄K₃K.

In some embodiments, X² of peptide multimers of antibiotic compositions provided herein is a lysine (K) and X⁴ is an arginine (R). In such embodiments, the peptide may comprise Formula II: [(R)ₐ(X)_{c}(K)_{b}(X)_{c}(R)ₐ(X)_{c}(K)_{b}]ₙ(K)ₙK. In some embodiments, X of Formula II is a glycine (G) or alanine (A). In some embodiment, X of Formula II is glycine (G). In some embodiments, X of Formula II is alanine (A). In some embodiments, a and b are independently selected to be an integer from 1 to 10. In some embodiments, c is an integer selected from 0 to 5. In some embodiments, n is at least one. In some embodiments, a and b may be independently selected from an integer from 1 to 10. In some embodiments, a and b may be the same or different from one another. In some embodiments, a may be 1, 2, 3, 4, 5, 6, 7, 8, 9 or 10. In some embodiments, b may be 1, 2, 3, 4, 5, 6, 7, 8, 9 or 10. As non-limiting examples, a may be 1 and b may be 1; a may be 1 and b may be 2; a may be 1 and b may be 3; a may be 1 and b may be 4; a may be 1 and b may be 5; a may be 1 and b may be 6; a may be 1 and b may be 7; a may be 1 and b may be 8; a may be 1 and b may be 9; a may be 1 and b may be 10; a may be 2 and b may be 1; a may be 3 and b may be 1; a may be 4 and b may be 1; a may be 5 and b may be 1; a may be 6 and b may be 1; a may be 7 and b may be 1; a may be 8 and b may be 1; a may be 9 and b may be 1; a may be 10 and b may be 1; a may be 2 and b may be 2; a may be 2 and b may be 3; a may be 2 and b may be 4; or any combination thereof. In some embodiments, c may be an integer selected from 0 to 5. For example, c may be 0, 1, 2, 3, 4 or 5.

In some embodiments, antibacterial compositions provided herein that include a peptide multimer and a carbapenem antibiotic (e.g., any of the carbapenem antibiotics described herein) include a carbapenem antibiotic and a peptide multimer of Formula II that is a dimer (e.g., having the formula

[(R)ₐ(X)_{c}(K)_{b}(X)_{c}(R)ₐ(X)_{c}(K)_{b}]₂KK).

In some embodiments, antibacterial compositions provided herein that include a peptide multimer and a carbapenem antibiotic (e.g., any of the carbapenem antibiotics described herein) include a carbapenem antibiotic and a peptide multimer of Formula II that is a trimer (e.g., having the formula

[(R)ₐ(X)_{c}(K)_{b}(X)_{c}(R)ₐ(X)_{c}(K)_{b}]KK[(K)_{b}(X)_{c}(R)ₐ(X)_{c}(K)_{b}(X)_{c}(R)ₐ]).

In some embodiments, antibacterial compositions provided herein that include a peptide multimer and a carbapenem antibiotic (e.g., any of the carbapenem antibiotics described herein) include a carbapenem antibiotic and a peptide multimer of Formula II that is a tetramer (e.g., having the formula

[(R)ₐ(X)_{c}(K)_{b}(X)_{c}(R)ₐ(X)_{c}(K)_{b}]₄(K)₃K).

In some embodiments of antibacterial compositions including a carbapenem antibiotic (e.g., any of the carbapenem antibiotics described herein) and a peptide multimer, the peptide multimer comprises Formula III:

[R(X⁵)_{d}RK(X⁶)ₑRR]ₙ(K)ₙ₋₁K.

In some embodiments, X⁵ and X⁶ may be the same or different from one another. In some embodiments, X⁵ and X⁶ are independently of each other an amino acid including, but not limited to, arginine (R), histidine (H), lysine (K), aspartic acid (D), glutamic acid (E), serine (S), threonine (T), asparagine (N), glutamine (Q), cysteine (C), glycine (G), proline (P) or alanine (A). In some embodiments, X⁵ and X⁶ are one of glycine (G), alanine (A) or arginine (R). In some embodiments, X⁵ is glycine (G) and X⁶ is glycine (G). In some embodiments, X⁵ is alanine (A) and X⁶ is glycine (G). In some embodiments, X⁵ is arginine (R) and X⁶ is glycine (G). In some embodiments, X⁵ is glycine (G) and X⁶ is alanine (A). In some embodiments, X⁵ is alanine (A) and X⁶ is alanine (A). In some embodiments, X⁵ is arginine (R) and X⁶ is alanine (A). In some embodiments, X⁵ is glycine (G) and X⁶ is arginine (R). In some embodiments, X⁵ is alanine (A) and X⁶ is arginine (R). In some embodiments, X⁵ is arginine (R) and X⁶ is arginine (R). In some embodiments, d and e are independently from each other an integer selected from 0 to 2. In some embodiments, d or e are 0, 1 or 2. In some embodiments, d is 0, 1 or 2. In some embodiments, e is 0, 1 or 2. For example, d may be 0 and e may be 0; d may be 0 and e may be 1; d may be 0 and e may be 2; d may be 1 and e may be 0; d may be 1 and e may be 1; d may be 1 and e may be 2; d may be 2 and e may be 0; d may be 2 and e may be 1; or d may be 2 and e may be 2.

In some embodiments of antibacterial compositions including a carbapenem antibiotic (e.g., any of the carbapenem antibiotics described herein) and a peptide multimer, the peptide multimer comprises Formula IV:

[RARK(X⁶)ₑRR]ₙ(K)ₙ₋₁K.

In some embodiments, e is an integer selected from 0 to 2. In some embodiment, e is 0, 1 or 2. In on some embodiments, n is at least one, is at least two, is at least three, or is four. In some embodiments, n is an integer selected from the group consisting of 1, 2, 3, 4, 5, 6, 7 or 8. In some embodiments, X⁶ is glycine (G) or alanine (A). In some embodiments, the peptide multimer of Formula IV is selected from the group consisting of: (RARKGGRR)₂KK (SEQ ID NO: 3), (RARKGRR)₂KK (SEQ ID NO: 4), (RARKARR)₂KK (SEQ ID NO: 5), (RARKAARR)₂KK (SEQ ID NO: 6) and (RARKRR)₂KK (SEQ ID NO: 7).

In some embodiments of antibacterial compositions including a carbapenem antibiotic (e.g., any of the carbapenem antibiotics described herein) and a peptide multimer, the peptide multimer comprises Formula V:

[RGRK(X⁶)ₑRR]ₙ(K)ₙ₋₁K.

In some embodiments, X⁶ is valine (V), glycine (G), or alanine (A). In some embodiments, the peptide multimer of Formula V is selected from the group consisting of: (RGRKGGRR)₂KK (SEQ ID NO: 8 (the peptide multimer of SEQ ID NO: 8 is also referred to herein and elsewhere as “B2088_99”, “B2088/99”, “G2D” and “G2D- dimer”), (RGRKGGRR)₂KK (SEQ ID NO: 9), (RGRKGRR)₂KK (SEQ ID NO: 10), (RGRKRR)₂KK (SEQ ID NO: 11), (RGRKAARR)₂KK (SEQ ID NO: 11), (RGRKARR)₂KK (SEQ ID NO: 13), (RGRKGGRR)₂KKRRGGKRGR (SEQ ID NO: 14), (RGRKGRR)₂KKRRGKRGR (SEQ ID NO: 15), (RGRKRR)₂KKRRKRGR (SEQ ID NO: 16) and (RGRKWRR)₂KK (SEQ ID NO: 17) (the peptide multimer of SEQ ID NO: 17 is also referred to herein and elsewhere as “B2088”, “V2D” and “V2D-dimer”).

In some embodiments of antibacterial compositions including a carbapenem antibiotic (e.g., any of the carbapenem antibiotics described herein) and a peptide multimer, the peptide multimer comprises Formula VI:

[RRKR]ₙ(K)ₙ₋₁K

In some embodiments, the peptide multimer of Formula VI is (RRKRR)₂KK (SEQ ID NO: 18) or (RRKRR)₂KKRRKRR (SEQ ID NO: 19).

In some embodiments of antibacterial compositions including a carbapenem antibiotic (e.g., any of the carbapenem antibiotics described herein) and a peptide multimer, the peptide multimer comprises Formula VII:

[(R)ₐ(K)_{b}(X)_{c}(R)ₐ(K)_{b}]ₙ(K)ₙ₋₁K.

In some embodiments, X of Formula VII may be a non-hydrophobic amino acid. In some embodiments, X of Formula VII is an amino acid other than a hydrophobic amino acid. For example, X of Formula VII can be arginine (R), histidine (H), lysine (K), aspartic acid (D), glutamic acid (E), serine (S), threonine (T), asparagine (N), glutamine (Q), cysteine (C), glycine (G), proline (P) or alanine (A). In some embodiments, X of Formula VII is an amino acid other than G or A. In some embodiments, a peptide multimer of Formula VII is selected from the group consisting of: [(R)ₐ(X)_{b}X_{c}(R)ₐ(X)_{b}]₂KK, [(R)ₐ(X)_{b}X_{c}(R)ₐ(X)_{b}]₂[(KKRK)_{b}(R)ₐX_{c}(K)_{b}(R)ₐ], and [(R)ₐ(X)_{b}X_{c}(R)ₐ(X)_{b}]₄K₃K.

In some embodiments of antibacterial compositions that include a carbapenem antibiotic (e.g., any of the carbapenem antibiotics described herein) and a peptide multimer, the peptide multimer comprises a peptide dimer, a peptide trimer, a peptide tetramer, a peptide pentamer, a peptide hexamer, a peptide heptamer, a peptide octamer, a peptide nonamer or a peptide decamer.

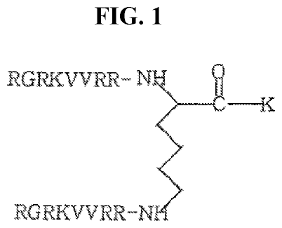

In some embodiments of antibacterial compositions including a carbapenem antibiotic (e.g., any of the carbapenem antibiotics described herein) and a peptide multimer, the peptide multimer comprises a peptide dimer. For example, a peptide dimer can be of the formula (U)₂, wherein U is a peptide monomer. In some embodiments, a peptide dimer is of the formula (U)₂Kₙ, wherein n is 1 or 2. In some embodiments, a peptide dimer of any of the variety of antibacterial compositions described herein is [RGRKVVRR)₂KK], also referred to as B2088. The structure of B2088 is shown in FIG. 1.

In some embodiments of antibacterial compositions that include a carbapenem antibiotic (e.g., any of the carbapenem antibiotics described herein) and a peptide dimer, the peptide dimer includes monomer subunits selected from the group consisting of: RGRKXXRR (SEQ ID NO: 20, wherein X is any amino acid), RGRKVVRR (SEQ ID NO: 21), RGRKAARR (SEQ ID NO: 22), RGRKGGRR (SEQ ID NO: 23), RGRKLLRR (SEQ ID NO: 24), RGRKIIRR (SEQ ID NO: 25), RGRKHHRR (SEQ ID NO: 26), RGRKWWRR (SEQ ID NO: 27), RGRKFFRR (SEQ ID NO: 28), and RGRKYYRR (SEQ ID NO: 29).

In some embodiments of antibacterial compositions that include a carbapenem antibiotic (e.g., any of the carbapenem antibiotics described herein) and a peptide dimer, the peptide dimer includes monomer subunits that are derivatives of RGRKVVRR (SEQ ID NO: 21) having one or more (e.g., 1, 2, 3, 4, 5, 6, 7, or 8) alanine substitutions. For example, a peptide dimer can have peptide monomer subunits selected from the group consisting of: AGRKVVRR (SEQ ID NO: 30), RARKVVRR (SEQ ID NO: 31), RGAKVVRR(SEQ ID NO: 32), RGRAVVRR (SEQ ID NO: 33), RGRKVARR (SEQ ID NO: 34), RGRKVARR (SEQ ID NO: 35), RGRKVVAR (SEQ ID NO: 36), RGRKVVRA (SEQ ID NO: 37), RGAAVVRR (SEQ ID NO: 38), RGRKVVAA (SEQ ID NO: 39), RGAKAVRR (SEQ ID NO: 40), RGRKAARR (SEQ ID NO: 41), RGAAAVRR (SEQ ID NO: 42), RGAKAARR (SEQ ID NO: 43), RGRAAARR (SEQ ID NO: 44), RGAAAARR (SEQ ID NO: 45), and RGRKAAAA (SEQ ID NO: 46). In some embodiments of peptide dimers that include monomer subunits that are alanine-substituted derivatives of RGRKVVRR, the alanine substitutions occur at one or more of amino acid positions 3 to 8 of RGRKVVRR (e.g., the monomers include an R at the first amino acid position and a G of the second amino acid position).

In some embodiments of antibacterial compositions that include a carbapenem antibiotic (e.g., any of the carbapenem antibiotics described herein) and a peptide dimer, the peptide monomers of the peptide dimer are the same (e.g., both of the peptide monomers has an identical amino acid sequence, e.g., one of SEQ ID NO: 20 to SEQ ID NO: 46). In some embodiments, the peptide monomers of the peptide dimer are different (e.g., one peptide monomer has a first amino acid sequence of one of SEQ ID NO: 20 to SEQ ID NO: 46, and the second peptide monomer has a second amino acid sequence of one of SEQ ID NO: 20 to SEQ ID NO: 46, wherein the first amino acid sequence of the first monomer and the second amino acid sequence of the second monomer are different).

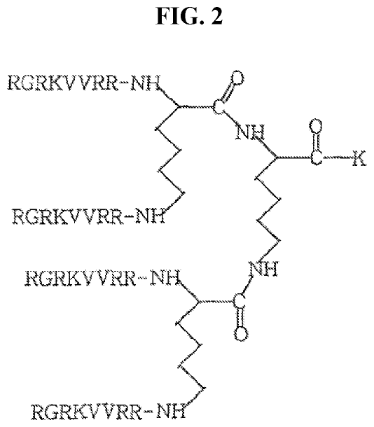

In some embodiments of antibacterial compositions including a carbapenem antibiotic (e.g., any of the carbapenem antibiotics described herein) and a peptide multimer, the peptide multimer comprises a peptide tetramer. For example, a peptide tetramer can be of the formula [(U)₂K]₂KKᵢ, wherein U is a peptide monomer and i=0 or 1. In some embodiments, a peptide tetramer of any of the variety of antibacterial compositions described herein is [RGRKVVRR)₂K]₂KK, also referred to as B4010. The structure of B4010 is shown in FIG. 2.

In some embodiments of antibacterial compositions that include a carbapenem antibiotic (e.g., any of the carbapenem antibiotics described herein) and a peptide tetramer, a peptide tetramer includes monomer subunits selected from the group consisting of: RGRKXXRR (SEQ ID NO: 20, wherein X is any amino acid), RGRKVVRR (SEQ ID NO: 21), RGRKAARR (SEQ ID NO: 22), RGRKGGRR (SEQ ID NO: 23), RGRKLLRR (SEQ ID NO: 24), RGRKIIRR (SEQ ID NO: 25), RGRKHHRR (SEQ ID NO: 26), RGRKWWRR (SEQ ID NO: 27), RGRKFFRR (SEQ ID NO: 28), and RGRKYYRR (SEQ ID NO: 29).

In some embodiments of antibacterial compositions that include a carbapenem antibiotic (e.g., any of the carbapenem antibiotics described herein) and a peptide tetramer, a peptide tetramer includes monomer subunits that are derivatives of RGRKVVRR (SEQ ID NO: 21) having one or more (e.g., 1, 2, 3, 4, 5, 6, 7, or 8) alanine substitutions. For example, a peptide tetramer can have peptide monomer subunits selected from the group consisting of: AGRKVVRR (SEQ ID NO: 30), RARKVVRR (SEQ ID NO: 31), RGAKVVRR(SEQ ID NO: 32), RGRAVVRR (SEQ ID NO: 33), RGRKVARR (SEQ ID NO: 34), RGRKVARR (SEQ ID NO: 35), RGRKVVAR (SEQ ID NO: 36), RGRKVVRA (SEQ ID NO: 37), RGAAVVRR (SEQ ID NO: 38), RGRKVVAA (SEQ ID NO: 39), RGAKAVRR (SEQ ID NO: 40), RGRKAARR (SEQ ID NO: 41), RGAAAVRR (SEQ ID NO: 42), RGAKAARR (SEQ ID NO: 43), RGRAAARR (SEQ ID NO: 44), RGAAAARR (SEQ ID NO: 45), and RGRKAAAA (SEQ ID NO: 46). In some embodiments of peptide tetramer that include monomer subunits that are alanine-substituted derivatives of RGRKVVRR, the alanine substitutions occur at one or more of amino acid positions 3 to 8 of RGRKVVRR (e.g., the monomers include an R at the first amino acid position and a G of the second amino acid position).

In some embodiments of antibacterial compositions that include a carbapenem antibiotic (e.g., any of the carbapenem antibiotics described herein) and a peptide tetramer, the peptide monomers of the peptide tetramer are the same (e.g., all four of the peptide monomers have an identical amino acid sequence, e.g., one of SEQ ID NO: 20 to SEQ ID NO: 46). In some embodiments, the peptide monomers of the peptide tetramer are different (e.g., at least one peptide monomer has a first amino acid sequence of one of SEQ ID NO: 20 to SEQ ID NO: 46, and at least one of the second, third, or fourth peptide monomers has a second amino acid sequence of one of SEQ ID NO: 20 to SEQ ID NO: 46, wherein the first amino acid sequence of the first monomer and the second amino acid sequence of the second, third or fourth monomer are different). In some embodiments, each of the four peptide monomers of the peptide tetramer are different from each other. In some embodiments, at least two of the four peptide monomers of the peptide tetramer are different from each other. In some embodiments, at least three of the four peptide monomers of the peptide tetramer are different from each other. In some embodiments, each of the four peptide monomers of the peptide tetramer are different from each other.

In some embodiments of antibacterial compositions that include a carbapenem antibiotic (e.g., any of the carbapenem antibiotics described herein) and a peptide multimer (e.g., any of the peptide multimers described herein), the peptide multimer may be chemically modified. As non-limiting examples, such a chemical modification may include amidation, acetylation, stapling, replacing at least one L-amino acid with a corresponding D-amino acid, introducing or replacing at least one amino acid with a non-natural amino acid (e.g., a non-proteogenic amino acid), and lipidation. As used herein, the term “lipidation” refers to modification that results in the covalent binding a lipid group to a peptide multimer. Lipidation may include, but is not limited to N- myristoylation, palmitoylation, GPI-anchor addition, prenylation, and other types of lipidation. For example, the lipid group present on a peptide multimer of antibacterial compositions provided herein can be CₘH2ₘ₋₁—CONH. In some embodiments, m can be an integer selected from 1 to 25 (e.g., 1, 2, 3, 4, 5, 6, 7, 8, 9, 10, 11, 12, 13, 14, 15, 16, 17, 18, 19, 20, 21, 22, 23, 24 or 25). For example, when m is 25, the lipid group is cerotic acid. The lipid group may also include cis or trans-form of unsaturated fatty acid with single or multi-double bonds, which can be synthetic or derived from nature (e.g., a fatty acid produced by microorganism).

The lipid group of the lipid-modified peptide multimer of antibacterial compositions provided herein can be coupled to the peptide multimer by using methods known in the art, for example using the solid phase peptide synthesis (SPPS). In brief, the general principle of SPPS is using repeated cycles of coupling-wash-deprotection-wash process. For example, peptide multimers can be immobilized on solid phase (e.g., example small solid beads or resins), which solid phase can be insoluble and/or porous. After immobilisation, the peptide multimers can be treated with functional units. The free N- terminal amine of the immobilised peptide multimer can then be coupled to a single N-protected amino acid unit. This unit can then be deprotected using appropriate reagent such as piperidine, revealing free N-terminal amine, which can be used to attach the next N-protected amino acid with free carboxylic group. The reaction mixture can be filtered in each step, and the peptide multimers can be immobilized on the beads or resins are retained during the filtration process, whereas liquid-phase reagents and by-products of synthesis can be flushed away. To synthesize the lipid-modified peptide multimers, instead of N-protected amino acid with free carboxylic acid, fatty acid with desired carbon length with free carboxylic acid can be used in coupling process. The reagent used in coupling process is similar to those used in coupling two amino acids. Before the peptide multimer is cleaved from the beads or resins by cleaving reagents such as trifluoroacetic acid (TFA), the growing peptide multimers can remain covalently attached to the beads or resins. After cleaving, the peptide multimers or lipid- modified peptide multimers can be collected and purified using high-performance liquid chromatography (HPLC).

Any of a variety of carbapenem antibiotics can be used in the methods described herein. As used herein, “carbapenem antibiotic” refers to antibiotically effective compounds comprising the structural element:

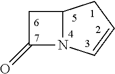

Capapenem antibiotics are defined as having the 4:5 fused ring lactam of penicillins with a double bond between C-2 and C-3 but with the substitution of carbon for sulfur at C-1 (see, e.g., K. M. Papp-Wallace et al., Antimicrob Agents Chemother. 2011 Nov.; 55(11): 4943-4960, which is incorporated by reference in its entirety herein). Suitable examples are described e.g. in A. Bryskier “Carbapenems”, Antimicrobial Agents: Antibacterials and Antifungals, page 270-321, Publisher: American Society for Microbiology, Washington D.C., 2005, and references cited therein. Non-limiting examples of carbapenem antibiotics include meropenem, imipenem, doripenem, eratapenem, panipenem (betamipron), biapenem, tebipenem, razupenem (PZ-601), lenapenem, tomopenem (thienpenem), ritipenem, sulopenem, ME1036 (formerly CP5609, a carbapenem with a 7-acylated imidazo[5,1-b]thiazole-2-yl group directly attached to the carbapenem moiety of the C-2 position), RO4908463, SM216601, or a pharmaceutically acceptable salt thereof. Additional examples of carbapenem antibiotics include those disclosed in WO 2008/116813, which is incorporated by reference in its entirety herein.

In some embodiments, any of the variety of antibacterial compositions provided herein include a single carbapenem antibiotic (e.g., any of the variety of carbapenem antibiotics disclosed herein). In embodiments of antibacterial compositions that include a single carbapenem antibiotic, the antibacterial composition can include a single or two or more (e.g., 2, 3, 4, 5, 6, 7, 8, 9, 10, or more) peptide multimers (e.g., any of the variety of peptide multimers disclosed herein). In some embodiments, any of the variety of antibacterial compositions provided herein include two or more (e.g., 2, 3, 4, 5, 6, 7, 8, 9, 10, or more) carbapenem antibiotics (e.g., any of the variety of carbapenem antibiotics disclosed herein). In embodiments of antibacterial compositions that include two or more carbapenem antibiotics, the antibacterial composition can include a single or two or more (e.g., 2, 3, 4, 5, 6, 7, 8, 9, 10, or more) peptide multimers (e.g., any of the variety of peptide multimers disclosed herein).

In some embodiments, antibacterial compositions provided herein include a peptide dimer and a carbapenem antibiotic (e.g., any of the variety of carbapenem antibiotics described herein or known in the art). Non limiting examples of antibacterial compositions that include a peptide dimer and a carbapenem antibiotic include: B2088 (e.g., lipidated or non-lipidated) and meropenem, B2088 (e.g., lipidated or non-lipidated) and imipenem, B2088 (e.g., lipidated or non-lipidated) and doripenem, B2088 (e.g., lipidated or non-lipidated) and eratapenem, B2088 (e.g., lipidated or non-lipidated) and panipenem, B2088 (e.g., lipidated or non-lipidated) and biapenem, B2088 (e.g., lipidated or non-lipidated) and tebipenem, B2088 (e.g., lipidated or non-lipidated) and razupenem, B2088 (e.g., lipidated or non-lipidated) and lenapenem, B2088 (e.g., lipidated or non-lipidated) and tomopenem, and B2088 (e.g., lipidated or non-lipidated) and tomopenem.

Other non-limiting examples of antibacterial compositions that include a peptide dimer and a carbapenem antibiotic include: a peptide dimer (e.g., lipidated or non-lipidated) having at least one monomer subunit (e.g., two monomer subunits) of any one of SEQ ID NO: 20-46 and meropenem, a peptide dimer (e.g., lipidated or non-lipidated) having at least one monomer subunit (e.g., two monomer subunits) of any one of SEQ ID NO: 20-46 and imipenem, a peptide dimer (e.g., lipidated or non-lipidated) having at least one monomer subunit (e.g., two monomer subunits) of any one of SEQ ID NO: 20-46 and doripenem, a peptide dimer (e.g., lipidated or non-lipidated) having at least one monomer subunit (e.g., two monomer subunits) of any one of SEQ ID NO: 20-46 and eratapenem, a peptide dimer (e.g., lipidated or non-lipidated) having at least one monomer subunit (e.g., two monomer subunits) of any one of SEQ ID NO: 20-46 and panipenem, a peptide dimer (e.g., lipidated or non-lipidated) having at least one monomer subunit (e.g., two monomer subunits) of any one of SEQ ID NO: 20-46 and biapenem, a peptide dimer (e.g., lipidated or non-lipidated) having at least one monomer subunit (e.g., two monomer subunits) of any one of SEQ ID NO: 20-46 and tebipenem, a peptide dimer (e.g., lipidated or non-lipidated) having at least one monomer subunit (e.g., two monomer subunits) of any one of SEQ ID NO: 20-46 and razupenem, a peptide dimer (e.g., lipidated or non-lipidated) having at least one monomer subunit (e.g., two monomer subunits) of any one of SEQ ID NO: 20-46 and lenapenem, a peptide dimer (e.g., lipidated or non-lipidated) having at least one monomer subunit (e.g., two monomer subunits) of any one of SEQ ID NO: 20-46 and tomopenem, and a peptide dimer (e.g., lipidated or non-lipidated) having at least one monomer subunit (e.g., two monomer subunits) of any one of SEQ ID NO: 20-46 and tomopenem. In some embodiments, the two peptide monomer subunits of the peptide dimer are the same. In some embodiments, the two peptide monomer subunits of the peptide dimer are different.

In some embodiments, antibacterial compositions provided herein include a peptide tetramer and a carbapenem antibiotic. Non-limiting examples of antibacterial compositions that include a peptide tetramer and a carbapenem antibiotic include: a peptide tetramer (e.g., lipidated or non-lipidated) having at least one monomer subunit (e.g., one, two, three, or four monomer subunits) of any one of SEQ ID NO: 20-46 and meropenem, a peptide tetramer (e.g., lipidated or non-lipidated) having at least one monomer subunit (e.g., one, two, three, or four monomer subunits) of any one of SEQ ID NO: 20-46 and imipenem, a peptide tetramer (e.g., lipidated or non-lipidated) having at least one monomer subunit (e.g., one, two, three, or four monomer subunits) of any one of SEQ ID NO: 20-46 and doripenem, a peptide tetramer (e.g., lipidated or non-lipidated) having at least one monomer subunit (e.g., one, two, three, or four monomer subunits) of any one of SEQ ID NO: 20-46 and eratapenem, a peptide tetramer (e.g., lipidated or non-lipidated) having at least one monomer subunit (e.g., one, two, three, or four monomer subunits) of any one of SEQ ID NO: 20-46 and panipenem, a peptide tetramer (e.g., lipidated or non-lipidated) having at least one monomer subunit (e.g., one, two, three, or four monomer subunits) of any one of SEQ ID NO: 20-46 and biapenem, a peptide tetramer (e.g., lipidated or non-lipidated) having at least one monomer subunit (e.g., one, two, three, or four monomer subunits) of any one of SEQ ID NO: 20-46 and tebipenem, a peptide tetramer (e.g., lipidated or non-lipidated) having at least one monomer subunit (e.g., one, two, three, or four monomer subunits) of any one of SEQ ID NO: 20-46 and razupenem, a peptide tetramer (e.g., lipidated or non-lipidated) having at least one monomer subunit (e.g., one, two, three, or four monomer subunits) of any one of SEQ ID NO: 20-46 and lenapenem, a peptide tetramer (e.g., lipidated or non-lipidated) having at least one monomer subunit (e.g., one, two, three, or four monomer subunits) of any one of SEQ ID NO: 20-46 and tomopenem, and a peptide tetramer (e.g., lipidated or non-lipidated) having at least one monomer subunit (e.g., one, two, three, or four monomer subunits) of any one of SEQ ID NO: 20-46 and tomopenem. In some embodiments, at least two (e.g., 2, 3 or 4) peptide monomer subunits of the peptide tetramer are the same. In some embodiments, at least two (e.g., 2, 3 or 4) peptide monomer subunits of the peptide tetramer are different.

### Bacteria

In some embodiments, a bacterium to be inhibited (e.g., in a subject having a bacterial infection, or outside the context of a medical treatment) with any of the variety of antibacterial compositions provided herein may be gram-positive or gram-negative. For example, a bacterium may be of genus including, but not limited to Acetobacter, Acinetobacter, Actinomyces, Agrobacterium spp., Azorhizobium, Azotobacter, Anaplasma spp., Bacillus spp., Bacteroides spp., Bartonella spp., Bordetella spp., Borrelia, Brucella spp., Burkholderia spp., Calymmatobacterium, Campylobacter, Chlamydia spp., Chlamydophila spp., Clostridium spp., Corynebacterium spp., Coxiella, Ehrlichia, Enterobacter, Enterococcus spp., Escherichia, Francisella, Fusobacterium, Gardnerella, Haemophilus spp., Helicobacter, Klebsiella, Lactobacillus spp., Lactococcus, Legionella, Listeria, Methanobacterium extroquens, Microbacterium multiforme, Micrococcus luteus, Moraxella catarrhalis, Mycobacterium spp., Mycoplasma spp., Neisseria spp., Pasteurella spp., Peptostreptococcus, Porphyromorias, Pseudomonas, Rhizobium, Rickettsia spp., Rochalimaea spp., Rothia, Salmonella spp., Serratia, Shigella, Staphylococcus spp., Stenotrophomonas, Streptococcus spp., Treponema spp., Vibrio spp., Wolbachia, and Yersinia spp. In some embodiments, a bacterium to be inhibited with any of the variety of antibacterial compositions provided herein may be Gram-positive or Gram-negative Acetobacter aurantius, Acinetobacter baumannii, Actinomyces israelii, Agrobacterium radiobacter, Agrobacterium tumefaciens, Azorhizobium caulinodans, Azotobacter vinelandii, Anaplasma phagocytophilum, Anaplasma marginate, Bacillus anthraces, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus (Prevotella melaminogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia complex, Burkholderia cenocepacia, Calymmatobacterium granulomatis, Campylobacter coli, Campylobacter fetus, Campylobacter jejuni, Campylobacter pylori, Chlamydia trachomatis, Chlamydophila. (such as C. pneumoniae, Chlamydophila psittaci, Clostridium botulinum, Clostridium difficile, Clostridium perfringens, Clostridium tetani), Corynebacterium diphtheriae, Corynebacterium fusiforme, Coxiella burnetii, Ehrlichia chqffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainjluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis Peptostreptococcus, Porphyromonas gingivalis, Pseudomonas aeruginosa, Rhizobium Radiobacter, Rickettsia prowazekii, Rickettsia psittaci, Rickettsia quintana, Rickettsia rickettsia, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus, avium, Streptococcus bovis, Streptococcus cricetus, Streptococcus faceium, Streptococcus faecalis, Streptococcus fetus, Streptococcus gallinarum, Streptococcus lactis, Streptococcus minor, Streptococcus mitis, Streptococcus mutans, Streptococcus oxalis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis, and Yersinia pseudotuberculosis.

In some embodiments, the bacterium is drug-resistant (e.g., carbapenem-resistant). For example, a bacterium that is carbapenem-resistant exhibits a minimum inhibitory concentration (MIC) for a carbapenem antibiotic of >1 μg/ml, e.g., >2 μg/ml, >3 μg/ml, >4 μg/ml, >5 μg/ml, >6 μg/ml, >7 μg/ml, or >8 μg/ml). In some embodiments, the bacterium is a gram negative bacteria such as a Pseudomonas (e.g., Pseudomonas aeruginosa), Escherichia (Escherichia coli), Acinetobacter (e.g., Acinetobacter baumanni) or Klebsiella (e.g., Klebsiella pneumoniae) species that is resistant to treatment with the carbapenem antibiotic. Non-limiting examples of drug-resistant (e.g., carbapenem-resistant) bacterial strains include various Klebsiella pneumonia strains such as KLPN 8852, KLPN 27025 and KLPN 24076, as well as various Acinetobacter baumannii strains such as ACBA 1010. Other bacteria that can develop drug resistance include, without limitation, Escherichia coli and Pseudomonas aeruginosa. In some embodiments, antibacterial compositions provided herein having a peptide multimer and a carbapenem antibiotic are more effective against a bacterium (e.g., a drug-resistant bacterium) than a reference antibacterial composition lacking either the peptide multimer or the carbapenem antibiotic. In some embodiments, antibacterial compositions provided herein having a peptide multimer and a carbapenem antibiotic are more effective against a bacterium (e.g., a drug-resistant bacterium) than a reference antibacterial composition lacking a peptide multimer but having a higher amount of the carbapenem antibiotic. In some embodiments, antibacterial compositions provided herein having a peptide multimer and a carbapenem antibiotic are more effective against a bacterium (e.g., a drug-resistant bacterium) than a reference antibacterial composition lacking a carbapenem antibiotic but having a higher amount of the peptide multimer.

In some embodiments, a bacterium to be eliminated and/or inhibited by any of the variety of antibacterial compositions provided herein may result in a bacterial infection when present in a subject. Such a bacterial infection can cause conditions such as, but not limited to, pneumonia, tuberculosis, meningitis, diarrhoeal diseases, formation of biofilm, sepsis, listeriosis, gastroenteritis, toxic shock syndrome, hemorrhagic colitis, hemolytic uremic syndrome, Lyme Disease, gastric and duodenal ulcers, human ehrlichiosis, pseudomembranous colitis, cholera, salmonellosis, cat scratch fever, necrotizing fasciitis (GAS), streptococcal toxic shock syndrome, nosocomial and community associated infections, atherosclerosis, sudden infant death syndrome (SIDS), wound infection, septicemia, gastrointestinal disease, hospital-acquired endocarditis and blood stream infections.

In some embodiments, B2088 and meropenem exhibit unexpected and synergistic antibacterial activity against strain KLPN 8852 (a carbapenemase resistant (“CRE”) strain of the common bacteria, KLPN, Klebsiella pneumonia). In some embodiments, B2088 and imipenem exhibit unexpected and synergistic antibacterial activity against strain KLPN 8852. In some embodiments, B2088 and doripenem exhibit unexpected and synergistic antibacterial activity against strain KLPN 8852.

In some embodiments, B2088 and meropenem exhibit unexpected and synergistic antibacterial activity against strain KLPN 27025 (a carbapenemase resistant (“CRE”) strain of KLPN). In some embodiments, B2088 and imipenem exhibit unexpected and synergistic antibacterial activity against strain KLPN 27025. In some embodiments, B2088 and imipenem exhibit unexpected and synergistic antibacterial activity against strain KLPN 8852. In some embodiments, B2088 and doripenem exhibit unexpected and synergistic antibacterial activity against strain KLPN 27025.

In some embodiments, B2088 and meropenem exhibit unexpected and synergistic antibacterial activity against strain KLPN 24076 (a carbapenemase resistant (“CRE”) strain of KLPN). In some embodiments, B2088 and imipenem exhibit unexpected and synergistic antibacterial activity against strain KLPN 24076. In some embodiments, B2088 and doripenem exhibit unexpected and synergistic antibacterial activity against strain KLPN 24076.

### REFERENCES

1. Susceptibility CLSI. 2014. Performance standards for antimicrobial testing: 24th informational supplement, CLSI document M100-S24. Clinical and Laboratory Standards Institute, Wayne, Pa.

2. The European Committee on Antimicrobial Susceptibility Testing. 2015. Breakpoint tables for interpretation of MICs and zone diameters. Version 5.0, 2015. http://www.eucast.org.

The above references are incorporated by reference herein in their entireties.

The invention will be further described in the following examples, which do not limit the scope of the invention described in the claims.

### EXAMPLES

### Example 1

KLPN 8852, 27025 and 24076 are carbapenemase resistant (“CRE”) strains of the common bacteria, KLPN, Klebsiella pneumonia. ACBA 1010 is a strain of Acinetobacter baumannii, a common hospital bacterium that often exhibits resistance. The ability of amikacin as well as three carbapenem antibiotics (meropenem, imipenem, and doripenem) to kill each of these strains was tested using minimum inhibitory concentration, or MIC, as a measure of effectiveness. Results are shown in Table 1 below.

TABLE 1
MIC of Amikacin, Imipenem, Doripenen, and Meropenem against
strains KPLN 8852, KPLN 27025, KPLN 24076, and ACBA 1010.

| | MIC dil in Full strength MHB | | | |
|---|---|---|---|---|
| | KLPN 8852 | KLPN 27025 | KLPN 24076 | ACBA 1010 |
| Amikacin | 6.25 | 0.78 | 1.56 | 1.56 |
| Imipenem | >50 | >50 | >50 | 25 |
| Doripenem | >50 | 25 | 25 | 0.195 |
| Meropenem | >50 | >50 | >50 | 1.56 |

As can be seen from Table 1, each of the carbapenem antibiotics exhibits an increase MIC against each of the tested Klebsiella pneumonia strains as compared to amikacin, indicating these strains are resistant to these carbapenem antibiotics.

### Example 2

### Synergy of B2088 with Carbapenem Antibiotics Against CRE Klebsiella pneumonia Strains

The ability of B2088 [RGRKVVRR)₂KK] to kill each of CRE strains KLPN 8852, 27025 and 24076 was tested by itself (as a monotherapy), as well as in combination with one of meropenem, imipenem, or doripenem (as a combination therapy) using minimum inhibitory concentration, or MIC, as a measure of effectiveness. Results are shown in Tables 2-4 below. Synergism MIC indicates the MIC of each of B2088 or the carbapenem antibiotic when used as an antibacterial composition.

TABLE 2
Synergism of B2088 with carbapenem antibiotics
against strain KLPN 8852.

| | | Monotherapy MIC | Synergy MIC |
|---|---|---|---|
| | | (μg/mL) | (μg/mL) |
| B2088 + | B2088 | 25 | 1 |
| Meropenem | Meropenem | >50 | 1 |
| B2088 + | B2088 | 25 | 0.5 |
| Imipenem | Imipenem | >50 | 0.5 |
| B2088 + | B2088 | 25 | 0.25 |
| Doripenem | Doripenem | >50 | 0.125 |

TABLE 3
Synergism of B2088 with carbapenem antibiotics
against strain KLPN 27025.

| | | Monotherapy MIC | Synergy MIC |
|---|---|---|---|
| | | (μg/mL) | (μg/mL) |
| B2088 + | B2088 | 6.25 | 0.0625 |
| Meropenem | Meropenem | 50 | 0.007813 |
| B2088 + | B2088 | 6.25 | 1 |
| Imipenem | Imipenem | >50 | 1 |
| B2088 + | B2088 | 6.25 | 0.015625 |
| Doripenem | Doripenem | 25 | 0.007813 |

TABLE 4
Synergism of B2088 with carbapenem antibiotics
against strain KLPN 24076.

| | | Monotherapy MIC | Synergy MIC |
|---|---|---|---|
| | | (μg/mL) | (μg/mL) |
| B2088 + | B2088 | 6.25 | 1 |
| Meropenem | Meropenem | >50 | 1 |
| B2088 + | B2088 | 6.25 | 1 |
| Imipenem | Imipenem | >50 | 1 |
| B2088 + | B2088 | 6.25 | 1 |
| Doripenem | Doripenem | 25 | 1 |

These data show that antibacterial compositions that include B2088 (a peptide multimers) and any of meropenem, imipenem, or doripenem (carbapenem antibiotics) exhibit unexpected and significantly improved antibacterial activity against CRE Klebsiella pneumonia strains as compared to B2088, meropenem, imipenem, or doripenem alone.

### Example 3

### Synergy of B2088 with Carbapenem Antibiotics Against ACBA 1010

The ability of B2088 [RGRKVVRR)₂KK] to kill strain ACBA 1010 was tested by itself (as a monotherapy), as well as in combination with one of meropenem, imipenem, or doripenem (as a combination therapy) using minimum inhibitory concentration, or MIC, as a measure of effectiveness. Results are shown in Table 5 below. Synergism MIC indicates the MIC of each of B2088 or the carbapenem antibiotic when used as an antibacterial composition.

TABLE 5
Synergism of B2088 with carbapenem antibiotics
against strain ACBA 1010.

| | | Monotherapy MIC | Synergy MIC |
|---|---|---|---|
| | | (μg/mL) | (μg/mL) |
| B2088 + | B2088 | 6.25 | 1 |
| Meropenem | Meropenem | 1.56 | 0.5 |
| B2088 + | B2088 | 6.25 | 1 |
| Imipenem | Imipenem | 12.5 | 1 |
| B2088 + | B2088 | 6.25 | 0.0625 |
| Doripenem | Doripenem | <0.0975 | 0.007813 |

These data show that antibacterial compositions that include B2088 (a peptide multimers) and any of meropenem, imipenem, or doripenem (carbapenem antibiotics) exhibit unexpected and significantly improved antibacterial activity against Acinetobacter baumannii strain ACBA 1010 as compared to B2088, meropenem, imipenem, or doripenem alone.

1. A method of inhibiting growth of a bacterium comprising:
contacting the bacterium with an effective amount of:
a peptide multimer of the formula (U)nBmZj, wherein U is a peptide comprising RGRKVVRR, wherein n≥2, m≥1, and j≥0, wherein each B comprises at least one amino acid having at least two amine groups, wherein Z comprises any amino acid, and wherein the multimer is branched at the terminal BmZj residues; and
a carbapenem antibiotic or a pharmaceutically acceptable salt thereof.

2. The method of claim 1, wherein the growth of the bacterium is inhibited by at least 2-fold as compared to a reference bacterium that is not contacted with the peptide multimer and the carbapenem antibiotic.

3. The method of claim 1, wherein the peptide multimer is present in an amount that is lower than the amount of the peptide multimer required to provide the same level of growth inhibition of the bacterium in the absence of the carbapenem antibiotic.

4. The method of claim 3, wherein the peptide multimer is present in an amount that is lower than about 4 μg/mL.

5. The method of claim 1, wherein the carbapenem antibiotic, or a pharmaceutically acceptable salt thereof, is selected from the group consisting of: meropenem, imipenem, doripenem, eratapenem, panipenem (betamipron), biapenem, tebipenem, razupenem (PZ-601), lenapenem, tomopenem, thienamycin (thienpenem), and combinations thereof.

6. The method of claim 1, wherein the carbapenem antibiotic, or a pharmaceutically acceptable salt thereof, is present in an amount that is lower than the amount of the carbapenem antibiotic, or a pharmaceutically acceptable salt thereof, required to provide the same level of growth inhibition of the bacterium in the absence of the peptide multimer.

7-16. (canceled)

17. A method of treating a bacterial infection in a subject comprising administering to the subject a therapeutically effective amount of:
a peptide multimer of the formula (U)nBmZj, wherein U is a peptide comprising RGRKVVRR, wherein n≥2, m≥1, and j≥0, wherein each B comprises at least one amino acid having at least two amine groups, wherein Z comprises any amino acid, and wherein the multimer is branched at the terminal BmZj residues; and
a carbapenem antibiotic or a pharmaceutically acceptable salt thereof.

18. The method of claim 17, wherein the number of bacteria is decreased in the subject by at least 2-fold as compared the number of bacteria in a reference subject that is not administered the peptide multimer and the carbapenem antibiotic.

19. The method of claim 17, wherein the peptide multimer is present in an amount that is lower than the amount of the peptide multimer required to provide the same level of bacterial inhibition when the peptide multimer is administered to the subject in the absence of the carbapenem antibiotic.

20. The method of claim 19, wherein the peptide multimer is administered in an amount from about 0.1 ug/ml and about 20 ug/ml.

21. The method of claim 17, wherein the carbapenem antibiotic, or a pharmaceutically acceptable salt thereof, is selected from the group consisting of: meropenem, imipenem, doripenem, eratapenem, panipenem (betamipron), biapenem, tebipenem, razupenem (PZ-601), lenapenem, tomopenem, thienamycin (thienpenem), and combinations thereof.

22. The method of claim 17, wherein the carbapenem antibiotic, or a pharmaceutically acceptable salt thereof, is administered in an amount that is lower than the amount of the carbapenem antibiotic, or a pharmaceutically acceptable salt thereof, required to provide the same level of bacterial inhibition when the carbapenem antibiotic is administered to a reference subject in the absence of the peptide multimer.

23-32. (canceled)

33. The method of claim 17, wherein the peptide multimer and the carbapenem antibiotic are administered to the subject simultaneously.

34. The method of claim 17, wherein the peptide multimer and the carbapenem antibiotic are administered to the subject sequentially.

35. An antibacterial composition comprising:
a peptide multimer of the formula (U)nBmZj, wherein U is a peptide comprising RGRKVVRR, wherein n≥2, m≥1, and j≥0, wherein each B comprises at least one amino acid having at least two amine groups, wherein Z comprises any amino acid, and wherein the multimer is branched at the terminal BmZj residues; and
a carbapenem antibiotic or a pharmaceutically acceptable salt thereof.

36. The composition of claim 35, wherein the carbapenem antibiotic, or a pharmaceutically acceptable salt thereof, is selected from the group consisting of: meropenem, imipenem, doripenem, eratapenem, panipenem (betamipron), biapenem, tebipenem, razupenem (PZ-601), lenapenem, tomopenem, thienamycin (thienpenem), and combinations thereof.

37. The composition of claim 36, wherein the peptide multimer comprises (RGRKVVRR)₂KK.

38. The composition of claim 35, wherein the carbapenem antibiotic comprises meropenem, and wherein the peptide multimer comprises (RGRKVVRR)₂KK.

39. The composition of claim 35, wherein the carbapenem antibiotic comprises imipenem, and wherein the peptide multimer comprises (RGRKVVRR)₂KK.

40. The composition of claim 35, wherein the carbapenem antibiotic comprises doripenem, and wherein the peptide multimer comprises (RGRKVVRR)₂KK.

41. (canceled)


SEQUENCE LISTING (46 sequences)

SEQ ID NO: 1 - protein, 45 aa, Artificial
  synthetic peptide
GIINTLQKYYCRVRGGRCAVLSCLPKEEQIGKCSTRGRKCCRRKK

SEQ ID NO: 2 - protein, 8 aa, Artificial
  synthetic peptide
RGRKVVRR

SEQ ID NO: 3 - protein, 18 aa, Artificial
  synthetic peptide
RARKGGRRRARKGGRRKK

SEQ ID NO: 4 - protein, 16 aa, Artificial
  synthetic peptide
RARKGRRRARKGRRKK

SEQ ID NO: 5 - protein, 16 aa, Artificial
  synthetic peptide
RARKARRRARKARRKK

SEQ ID NO: 6 - protein, 18 aa, Artificial
  synthetic peptide
RARKAARRRARKAARRKK

SEQ ID NO: 7 - protein, 14 aa, Artificial
  synthetic peptide
RARKRRRARKRRKK

SEQ ID NO: 8 - protein, 18 aa, Artificial
  synthetic peptide
RGRKGGRRRGRKGGRRKK

SEQ ID NO: 9 - protein, 18 aa, Artificial
  synthetic peptide
RGRKGGRRRGRKGGRRKK

SEQ ID NO: 10 - protein, 16 aa, Artificial
  synthetic peptide
RGRKGRRRGRKGRRKK

SEQ ID NO: 11 - protein, 14 aa, Artificial
  synthetic peptide
RGRKRRRGRKRRKK

SEQ ID NO: 12 - protein, 18 aa, Artificial
  synthetic peptide
RGRKAARRRGRKAARRKK

SEQ ID NO: 13 - protein, 16 aa, Artificial
  synthetic peptide
RGRKARRRGRKARRKK

SEQ ID NO: 14 - protein, 26 aa, Artificial
  synthetic peptide
RGRKGGRRRGRKGGRRKKRRGGKRGR

SEQ ID NO: 15 - protein, 23 aa, Artificial
  synthetic peptide
RGRKGRRRGRKGRRKKRRGKRGR

SEQ ID NO: 16 - protein, 20 aa, Artificial
  synthetic peptide
RGRKRRRGRKRRKKRRKRGR

SEQ ID NO: 17 - protein, 16 aa, Artificial
  synthetic peptide
RGRKWRRRGRKWRRKK

SEQ ID NO: 18 - protein, 12 aa, Artificial
  synthetic peptide
RRKRRRRKRRKK

SEQ ID NO: 19 - protein, 17 aa, Artificial
  synthetic peptide
RRKRRRRKRRKKRRKRR

SEQ ID NO: 20 - protein, 8 aa, Artificial
  synthetic peptide
  misc_feature (5)..(6): Xaa can be any naturally occurring amino acid
RGRKXXRR

SEQ ID NO: 21 - protein, 8 aa, Artificial
  synthetic peptide
RGRKVVRR

SEQ ID NO: 22 - protein, 8 aa, Artificial
  synthetic peptide
RGRKAARR

SEQ ID NO: 23 - protein, 8 aa, Artificial
  synthetic peptide
RGRKGGRR

SEQ ID NO: 24 - protein, 8 aa, Artificial
  synthetic peptide
RGRKLLRR

SEQ ID NO: 25 - protein, 8 aa, Artificial
  synthetic peptide
RGRKIIRR

SEQ ID NO: 26 - protein, 8 aa, Artificial
  synthetic peptide
RGRKHHRR

SEQ ID NO: 27 - protein, 8 aa, Artificial
  synthetic peptide
RGRKWWRR

SEQ ID NO: 28 - protein, 8 aa, Artificial
  synthetic peptide
RGRKFFRR

SEQ ID NO: 29 - protein, 8 aa, Artificial
  synthetic peptide
RGRKYYRR

SEQ ID NO: 30 - protein, 8 aa, Artificial
  synthetic peptide
AGRKVVRR

SEQ ID NO: 31 - protein, 8 aa, Artificial
  synthetic peptide
RARKVVRR

SEQ ID NO: 32 - protein, 8 aa, Artificial
  synthetic peptide
RGAKVVRR

SEQ ID NO: 33 - protein, 8 aa, Artificial
  synthetic peptide
RGRAVVRR

SEQ ID NO: 34 - protein, 8 aa, Artificial
  synthetic peptide
RGRKVARR

SEQ ID NO: 35 - protein, 8 aa, Artificial
  synthetic peptide
RGRKVARR

SEQ ID NO: 36 - protein, 8 aa, Artificial
  synthetic peptide
RGRKVVAR

SEQ ID NO: 37 - protein, 8 aa, Artificial
  synthetic peptide
RGRKVVRA

SEQ ID NO: 38 - protein, 8 aa, Artificial
  synthetic peptide
RGAAVVRR

SEQ ID NO: 39 - protein, 8 aa, Artificial
  synthetic peptide
RGRKVVAA

SEQ ID NO: 40 - protein, 8 aa, Artificial
  synthetic peptide
RGAKAVRR

SEQ ID NO: 41 - protein, 8 aa, Artificial
  synthetic peptide
RGRKAARR

SEQ ID NO: 42 - protein, 8 aa, Artificial
  synthetic peptide
RGAAAVRR

SEQ ID NO: 43 - protein, 8 aa, Artificial
  synthetic peptide
RGAKAARR

SEQ ID NO: 44 - protein, 8 aa, Artificial
  synthetic peptide
RGRAAARR

SEQ ID NO: 45 - protein, 8 aa, Artificial
  synthetic peptide
RGAAAARR

SEQ ID NO: 46 - protein, 8 aa, Artificial
  synthetic peptide
RGRKAAAA